(19) United States
(12) Patent Application Publication
(10) Pub. No.: US 2010/0076380 A1
(43) Pub. Date: Mar. 25, 2010

(54) BIOREACTORS

(75) Inventor: Mizhou Hui, Thousand Oaks, CA (US)

Correspondence Address: OCCHIUTI ROHLICEK & TSAO, LLP, 10 FAWCETT STREET, CAMBRIDGE, MA, 02138, US

(73) Assignees: AmProtein Corporation, Camarillo, CA (US); Hangzhou AmProtein Bioengineering Co., Ltd., Hangzhou (CN)

(21) Appl. No.: 12/485,841

(22) Filed: Jun. 16, 2009

Related U.S. Application Data
(60) Provisional application No. 61/132,190, filed on Jun. 16, 2008.
(60) Provisional application No. 61/192,515, filed on Sep. 19, 2008.
(60) Provisional application No. 61/216,193, filed on May 14, 2009.
(60) Provisional application No. 61/183,387, filed on Jun. 2, 2009.

(51) Int. Cl. A61M 1/10; C12M 1/00; C12M 1/34; C12N 5/00

(52) U.S. Cl. 604/151; 435/289.1; 435/287.1; 435/404

(57) ABSTRACT

Disclosed are various examples of methods and systems for providing an oxygenated culture environment for growing animal cells, as well as methods and systems for oxygenating blood and related methods for treating diseases and related conditions.

### CROSS-REFERENCE TO RELATED APPLICATIONS

This application claims the benefit of U.S. Provisional Application No. 61/183,387, titled “Novel Bioreactors,” filed Jun. 2, 2009, U.S. Provisional Application No. 61/216,193, titled “Blood Culture Bioreactor,” filed May 14, 2009, U.S. Provisional Application No. 61/192,515, titled “Novel Bioreactors,” filed Sep. 19, 2008, and U.S. Provisional Application No. 61/132,190, titled “Novel Bioreactors,” filed Jun. 16, 2008. The content of each of the above four applications is incorporated herein by reference in its entirety.

### BACKGROUND

Oxygen is a crucial nutrient for biological organisms. At the cell level, the survival of many types of cells relies on a continuous supply of oxygen in their growth environment. At the organ level of an animal, the lack of oxygen in the blood, even for brief periods, may cause organ dysfunction that could lead to catastrophic events such as brain damage, lung failure, or even death. There is a need for creating an oxygenated environment for culturing animal cells and a need for providing oxygenated blood for treating related diseases.

### SUMMARY

This application relates to systems and methods for culturing cells and for oxygenating blood.

One general aspect of the invention features a cell culture apparatus that includes a rotatable vessel having a wall for defining a culture chamber, and an inlet formed in the wall enabling fluid communication between the culture chamber and an external fluid source. In some examples, the wall has an inner surface configured to promote gas-inner surface contact. An actuator is coupled to the vessel for rotating the vessel about a first axis to increase the level of dissolved oxygen in a fluid in the culture chamber.

Embodiments of this aspect may include one or more of the following features.

The vessel has an outlet enabling fluid communication between the culture chamber and a sensor. The sensor can be used for measuring a fluid condition in the culture chamber, for example, the level of dissolved oxygen or the pH level of the fluid in the culture chamber.

A controller is provided for receiving a signal from the sensor and for determining the speed of rotation according to the received signal.

The external fluid source includes a liquid source and a gas source. A valve is provided in the system for selectively coupling the liquid source and the gas source to the inlet of the rotatable vessel. The liquid source can include a culture medium. The gas source can include one or more of the following: air, oxygen, carbon dioxide, and nitrogen.

A housing is provided for coupling the vessel to the actuator. A movable platform can be coupled to the housing for translating the vessel along a second axis.

The inner surface of the wall is characterized by micro-structures or nano-structures.

The vessel can be disposed in an at least partially closed environment (e.g., an incubator) of controlled temperature or controlled gaseous composition.

Another general aspect of the invention features a method for oxygenating a culture medium, for example, performed using the cell culture apparatus described above. The method includes introducing a culture medium into a culture chamber at least partially filled with gas, wherein the culture chamber is defined by a wall of a vessel, and an inner surface of the wall is configured to promote gas-inner surface contact. The method also includes rotating the vessel to create a relative movement of the culture medium with respect to the inner surface of the wall of the vessel to increase the level of dissolved oxygen in the culture medium.

Embodiments of this method may include one or more of the following features.

In rotating the vessel, microscopic bubbles carrying oxygen molecules are generated.

The method can also include measuring a fluid condition of the culture medium and controlling a speed of rotation according to the measured fluid condition.

The fluid condition includes one or more of the following: a pH level, a dissolved oxygen level, a carbon dioxide level, and a temperature.

A third general aspect of the invention features a culture system that has an oxygenation apparatus configured for oxygenating a culture medium. The oxygenation apparatus includes a vessel having an inlet for receiving the culture medium to be oxygenated, an outlet for providing oxygenated culture medium, and a wall with an inner surface configured to promote gas-inner surface contact; and an actuator coupled to the vessel for rotating the vessel to increase the level of dissolved oxygen in the culture medium. A culture apparatus is configured for receiving the oxygenated culture medium from the oxygenation apparatus. The culture apparatus includes a port for receiving a biological material into an interior of the culture apparatus; and an inlet in fluid communication with the interior of the culture apparatus to deliver the oxygenated culture medium into contact with the biological material.

Embodiment of this aspect may include one or more of the following features.

The biological material received in the culture apparatus includes cells or tissues.

The culture apparatus includes a perfusion column, which may be configured to receive a bio-compatible matrix suitable for cell culture. The bio-compatible matrix includes, for example, a microcarrier and a paper carrier. Some of these matrixes include polymer fibers, which can be woven or non-woven.

The flow rate of the culture medium exiting the perfusion column is controlled based on a height of the perfusion column. The flow rate of the culture medium entering the perfusion column is controlled to be substantially equal to the flow rate of the culture medium exiting the perfusion column. In one embodiment, the perfusion column is mounted on an adjustable stand configured for adjusting the height of the perfusion column.

The system also includes a pump for generating a flow of the oxygenated culture medium to be received by the culture apparatus.

A controller can be used for controlling a speed of the flow of the oxygenated culture medium, and can be further used to control a rotational or an orbital movement speed of the vessel of the oxygenation apparatus.

In some examples, the vessel of the oxygenation apparatus has an inverted frusto-conical bottom or cone-shaped round bottom.

A fourth general aspect of the invention features a culture system that includes a housing having a first terminus and a second and close terminus opposed to the first terminus, the first terminus having a port for receiving and dispensing a culture medium. A tubular member is disposed within an interior of the housing. The tubular member has a first open end; a second and close end positioned at the second terminus of the housing; and a disk membrane coupled to the first open end for at least partially enclosing an interior of the second tubular member to define a culture chamber, wherein the disk member includes at least an opening enabling fluid communication between the culture chamber and an exterior of the second tubular member. An actuator is coupled to the housing for rotating the housing to increase the level of dissolved oxygen in a fluid in the culture chamber.

Embodiments of this aspect may include one or more of the following features.

The tubular member is detachably disposed within the interior of the housing, and configured to receive a bio-compatible matrix suitable for cell culture through its first open end. An inner surface of the tubular member may be configured to promote gas-inner surface contact.

A fifth general aspect of the invention features a cell culture apparatus that includes a tubular member having a first end and a second and close end opposed to the first end, the first end having a port for receiving and dispensing a culture medium; a bio-compatible matrix disposed within an interior of the tubular member; and a disk member detachably disposed within the interior of the tubular member for substantially confining the bio-compatible matrix within a culture chamber defined between the disk member and the second end of the tubular member. The disk member includes at least an opening for permitting a flow of the culture medium into the culture chamber.

In one embodiment, a cap may be coupled to the first end of the tubular member for sealingly enclosing the interior of the tubular member.

A sixth general aspect of the invention features a cell culture system that includes an elongated member having a first close end; a second close end opposed to the first close end; a wall defining a culture chamber at least partially filled with gas; and an inlet formed in the wall capable of receiving a culture medium. A shaft is positioned substantially within the culture chamber, with its first end and second end respectively positioned at a corresponding end of the elongated member. A set of one or more disk elements is concentrically mounted on the shaft. An actuator is mechanically coupled to the shaft for rotating the disk elements about a longitudinal axis of the shaft for inducing a relative movement of the culture medium with respect to an inner surface of the wall of the elongated member. A sensor detects the level of dissolved oxygen in the culture medium contained in the culture chamber.

Embodiments of this aspect may include one or more of the following features.

A bio-compatible matrix can be disposed at a lower section of the interior of the elongate member. The bio-compatible matrix may include polymer fibers.

A controller is provided for receiving a signal from the sensor indicative of the level of dissolved oxygen in the culture medium, and further configured for controlling a speed of rotation of the shaft based on the received signal from the sensor.

The elongated member further includes a second inlet for introducing a flow of gas into the interior of the elongated member.

The controller is also configured for controlling a gaseous composition in the interior of the elongated member.

A seventh general aspect of the invention features a system for oxygenating blood and delivering the oxygenated blood to a subject. The system includes (1) a blood pump assembly adapted for coupling to a supply of blood; (2) a blood oxygenation assembly coupled to the blood pump assembly; (3) a delivery assembly; and (4) a control assembly.

The blood oxygenation assembly can receive blood from the blood pump assembly and oxygenate the blood. In one example, the blood oxygenation assembly has a blood bioreactor/oxygenator that includes (a) a housing including a wall; (b) a first inlet in the housing adapted to deliver oxygen-containing gas stream into contact with a first surface of the wall; (c) a second inlet in the housing adapted to deliver the blood into contact with a second surface of the wall, wherein the blood bioreactor/oxygenator allows a movement of the blood so that the blood contacts with the first surface to form oxygenated blood; and (d) a first outlet and a second outlet in the housing for expelling CO₂-containing gas and the oxygenated blood, respectively. The delivery assembly is coupled to the second outlet and is adapted to deliver the oxygenated blood to the subject; and the control assembly is coupled to the blood oxygenation assembly.

In one embodiment, the blood bioreactor/oxygenator allows the movement of the blood therein so that the blood and the oxygen-containing gas repeatedly and consecutively contact the first surface. For example, the movement can be a circular movement. In another embodiment, the housing defines a chamber that includes an inverted frusto-conical bottom or cone-shaped round bottom configured lower section.

In the above-described system, the blood bioreactor/oxygenator can further include a pH probe, a temperature probe, or a dissolved oxygen (DO) probe. In one embodiment, the system can also include a treatment assembly that removes an unwanted agent from the blood. Examples of such unwanted agent include a chemical agent, a bio molecule, an antibody, a virus, or a cell. In another embodiment, the system also includes a light/radiation source for killing an unwanted cell in the blood.

Further aspects of the invention provide a method for oxygenating blood of a subject (human or none-human animal) using the above-described system and a method can be used to remove an unwanted agent from the blood of a subject.

The invention also features a method of using the above-describe system for treating a subject having a disorder characterized with defect in blood oxygenation. Examples of the disorders include cardiovascular disorders (e.g., myocardial infarction), lung failure, or other respiratory disorders such as acute respiratory distress syndrome (ARDS). The invention also features methods for treating a subject having cancer and tumor metastasis, such as leukaemia, lymphoma, and sarcoma. The invention also features methods for treating a subject having an infection with a microbe, such as a bacterial cell, a yeast cell, or a virus. Each of the method include a step of removing the unwanted agent (e.g., cancer cell, bacterial cell, a yeast cell, or a virus) from the blood using the above-describe system.

Other features and advantages of the invention are apparent from the following description, and from the claims.

### DESCRIPTION OF DRAWINGS

FIG. 1 is a schematic diagram of a first embodiment of a cell culture system with a rotating bioreactor.

FIGS. 2A and 2B are schematic diagrams of one example of the bioreactor of FIG. 1.

FIG. 3 is an image of an inner surface of the bioreactor of FIG. 2A.

FIG. 4 is a schematic diagram of a second embodiment of a cell culture system with rotating bioreactor.

FIG. 5 is a photo of a third embodiment of a cell culture system with rotating bioreactors.

FIG. 6 is a schematic diagram of a fourth embodiment of a cell culture system with a rotating bioreactor.

FIGS. 7A and 7B are photos of a fifth embodiment of a cell culture system.

FIGS. 8A-8C are schematic diagrams of three embodiments of a perfusion cell culture system with an oxygenation assembly (“artificial lung”) and a paper carrier containing column (A and B) or a paper carrier containing culture vessel with a frusto-conical bottom (C).

FIG. 9 is a photo of another embodiment of a cell culture system with an oxygenation assembly installed in a clean chamber.

FIG. 10 is a photo of one example of a paper carrier-containing column for use with the perfusion bioreactor system of FIG. 9 and FIGS. 8A-8C.

FIG. 11 is a photo of one example of culture vessel's inner single-use plastic bag and for use as the bioreactor of FIG. 9 and FIGS. 8A-8C.

FIG. 12 is an electronic microscope image of the paper carrier inside the culture vessel of FIG. 10.

FIG. 13 is a photo of a second embodiment of a cell culture perfusion system with a classical impellor and air-bubbling-based bioreactor as an oxygenation assembly.

FIG. 14 is a schematic diagram of a first embodiment of a cell culture system with an oxygen transferring rotating wheel.

FIG. 15 is a schematic diagram of a second embodiment of a cell culture system with an oxygen transferring rotating wheel and stacked paper-carriers inside the culture vessel.

FIG. 16 is a block diagram of a first embodiment of a blood oxygenation system.

FIG. 17 is a schematic diagram of the first embodiment of the blood oxygenation, culture and re-circulation system of FIG. 16.

FIG. 18 is a schematic diagram of a second embodiment of a blood oxygenation, culture and re-circulation system of FIG. 16.

FIG. 19 is a photo of the second embodiment of the blood oxygenation in use.

FIG. 20 is an image of microcarrier-attached CHO cells in an oxygenated environment.

FIG. 21 is an image of microcarrier-attached VERO cells in an oxygenated environment.

FIGS. 22A and B are images of CHO-S (A) and CHO-K1 (B) cells growing in the cell culture system of FIG. 8.

FIGS. 23 A and B are images of MDCK (A) and VERO(B) cells growing in the cell culture system of FIG. 8A or B.

FIGS. 24 A and B are images of BHK-21(A) and MARC 145 (B) cells growing in the cell culture system of FIG. 8A or B.

### DETAILED DESCRIPTION

### 1 Overview

Oxygen is an essential nutrient for cells. Oxygen deprivation for even brief periods of time may result in cell damage, which may lead to organ dysfunction or failure. For example, heart attack and stroke victims experience blood flow obstructions or diversions that prevent oxygen from being delivered to the cells of vital tissues. Also, without oxygen, the heart and brain progressively deteriorate. Severe cases of oxygen deprivation can result in complete organ failure and even death. Less severe cases typically involve costly hospitalization, specialized treatments, and lengthy rehabilitation. In many biological and clinical settings, the level of oxygen in a culture medium or blood is closely monitored by measuring the concentration of dissolved oxygen (DO), i.e., the relative amount of oxygen dissolved or carried in a medium.

This application discloses various systems and techniques for oxygenating a cell culture environment and/or blood, for example, by increasing the DO level in a liquid solution, without necessarily using the conventional air bubbling or sparging techniques. These systems and techniques have a wide range of biological and medical applications.

### 2 Cell Culture System

This section describes various embodiments of a cell culture system capable of providing an oxygenated culture medium for use in many types of cell culture schemes.

2.1 System with Rotating Bioreactor

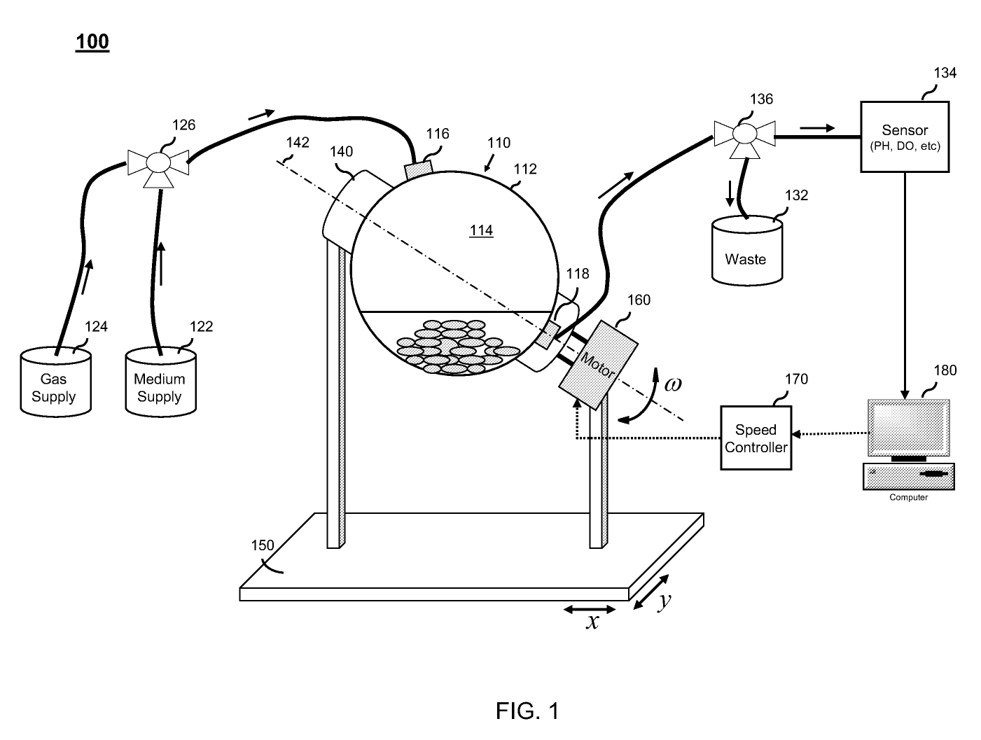

FIG. 1 shows a first embodiment of a cell culture system 100 that employs a rotatable bioreactor 110. This bioreactor can be used for growing cells in a suspension or adherent culture. In some examples, to promote the growth of adherent cells, a support matrix can be disposed inside the bioreactor 110, allowing cells to form attachment to the surface of the matrix. Examples of support matrix include a microcarrier (e.g., a matrix made from gelatin, collagen, cellulose, or other materials), and a paper carrier (e.g., a matrix made of polymer fibers and acrylic acid glue).

In this embodiment, the bioreactor 110 includes a wall 112 for defining a culture chamber 114 in which cells or tissues can grow. An inlet 116 is formed in the wall 112 to allow the passage of a culture medium 122 or a gas 124 into the culture chamber 114 via the use of a three-way valve 126. Depending on the particular application, the gas 124 may contain air, oxygen, nitrogen, carbon dioxide, or a combination thereof. An outlet 116 is also formed in the wall 112 to allow the culture medium inside the chamber 114 to be disposed to a waste collection 132, or alternatively, be tested by a sensor 134 (e.g., a pH probe, a DO probe, and a temperature probe).

The bioreactor 110 can be mounted onto a housing 140 mechanically coupled to a platform 150. An actuator 160 (e.g., a rotary motor) is coupled to the housing 140 for causing the bioreactor 110 to rotate or roll about an axis 142 at a speed ω, which may be controlled by a speed controller 170. The platform 150 may be stationary, or instead be movable along the x and y axes (shown in the figure), which can in turn cause cyclic movement of the bioreactor 110 along those two directions.

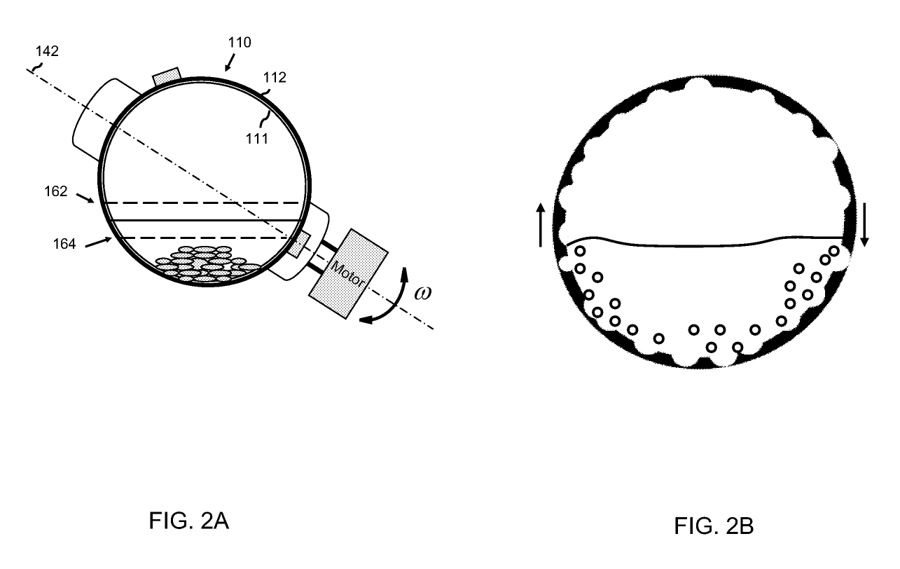

Referring to FIGS. 2A and 2B, as the bioreactor 110 rotates or rolls about axis 142, and/or moves along the x and y axes, the culture medium inside the chamber 114 repeatedly “sweeps” the inner surface of the wall, for example, between surface lines 162 and 164. In some examples, the recurrent medium-surface interactions caused by the movement of the bioreactor 110 generate an increased number of microscopic bubbles containing oxygen molecules. As these oxygen molecules dissolve into the culture medium, a high level of DO can be achieved. In some implementations, the above described technique can be used to increase the DO levels of culture mediums in a more efficient manner than directly bubbling air into the medium (such as the conventional sparging techniques).

In some examples, the bioreactor 110 is made of a glass or plastic container having a smooth inner surface. The bioreactor can be made in either disposable or reusable forms. In some examples, the bioreactor 110 includes a disposable container 111 (e.g., a plastic bag) positioned inside an autoclavable container (e.g., a glass housing).

In some examples, to further increase the oxygenation performance or to support high-density cell culture, the bioreactor 110 is made of materials with micro- or nano-level surface features. For example, the bioreactor of FIG. 2B has an uneven inner surface (e.g., with a set of concavities of various dimensions and shapes) for promoting gas-surface contact. In the first half of a rotational cycle when the culture medium climbs to one side of the inner surface of the bioreactor, a microscopic layer of oxygen bubbles can be formed at the other side of the surface that is instantly exposed to air (or gas). This microscopic bubble layer is then “washed” into the culture medium in the second half of the rotational cycle, causing oxygen to dissolve at a faster rate. In some examples, this effect can also be achieved with bioreactors of smooth inner surface.

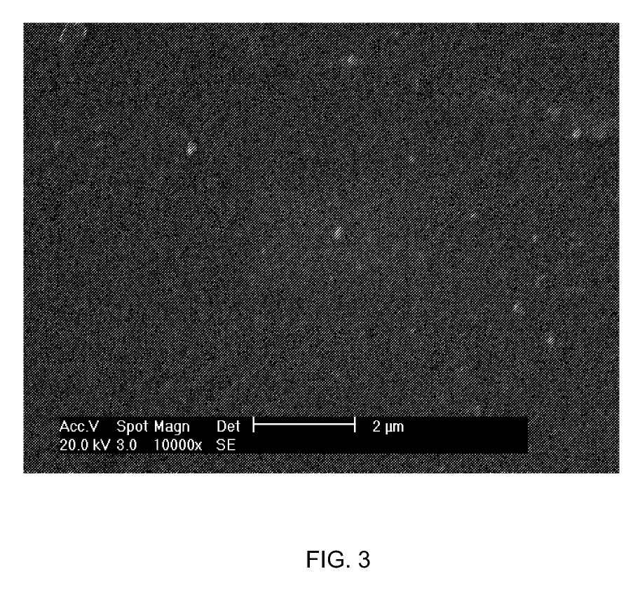

FIG. 3 shows an image of one type of inner surface suitable for high density cell culture and for increasing oxygenation.

In some examples, the wall of the bioreactor 110 is configured to feature “hilly” surface with microscopic surface details discernable at nanometer resolution (e.g., through electron microscopy). Such configurations may provide improved oxygen transfer characteristics in certain applications. For example, microscopic hills (or concavities) formed at 1˜2000 nanometers (e.g., 5, 10, 50, 100, 200, 500, 1000, or 1500 nanometers) in diameter can break the surface tension of the culture medium in contact with the wall, thereby generating microscopic bubbles. The wall of the bioreactor can be made from various materials, including for example glass, silica glass, steel, aluminum, array, and different types of plastics. It should be noted that even though the bubbles generated during the rotation of the bioreactor may not always be uniform in size or in spatial distribution, the presence of a significant portion of microscopic bubbles containing oxygen molecules can still result in an increase in the DO level of the culture medium.

Note that the rotational or rolling speed of the bioreactor 110 can be controlled by actively monitoring the DO level of the culture medium in the bioreactor. For example, a DO sensor 134 may be employed to detect the DO level in real time and report the measurement to a computer 180. The computer 180 compares the actual DO level with a target level to determine whether to increase or decrease the rotational speed of the motor 160. In some examples, the rotation of the bioreactor is controlled such that the culture medium inside the bioreactor appears to stay relative static, thereby reducing or limiting the impact of hydro-mechanical stress (e.g., flow-induced shear stress) on cells attached to a matrix. This can be a desirable feature in some applications, as impeller-based bioreactors tend to generate shear forces or agitation forces that may damage cells in a suspension culture. In some further examples, the rotational or rolling speed of the bioreactor is also determined based on the desired amount of sweeping force (or the desired speed of in-chamber medium current), which may correspond to the particular surface characteristics of the bioreactor.

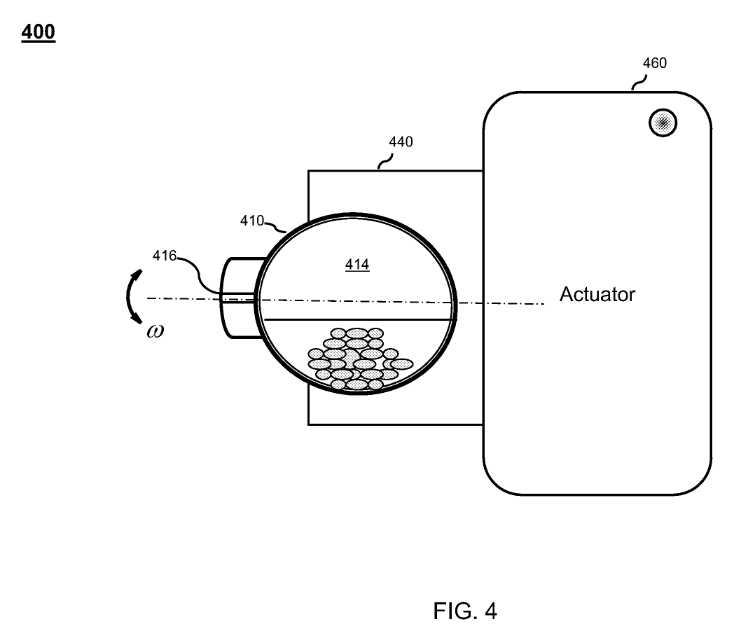

FIG. 4 shows a second embodiment of a cell culture system 400 that employs a rotatable bioreactor 410. Similar to the first embodiment, the bioreactor 410 is coupled to a housing 440 that can be driven into rotational or rolling movement by an actuator 460. The reactor 410 includes a wall 412 for defining a culture chamber 414 in which cells may grow. An inlet 416 is formed in the wall to allow passage of a culture medium, a gas, and/or a biological material (including cells and tissues) into and out of the culture chamber 414.

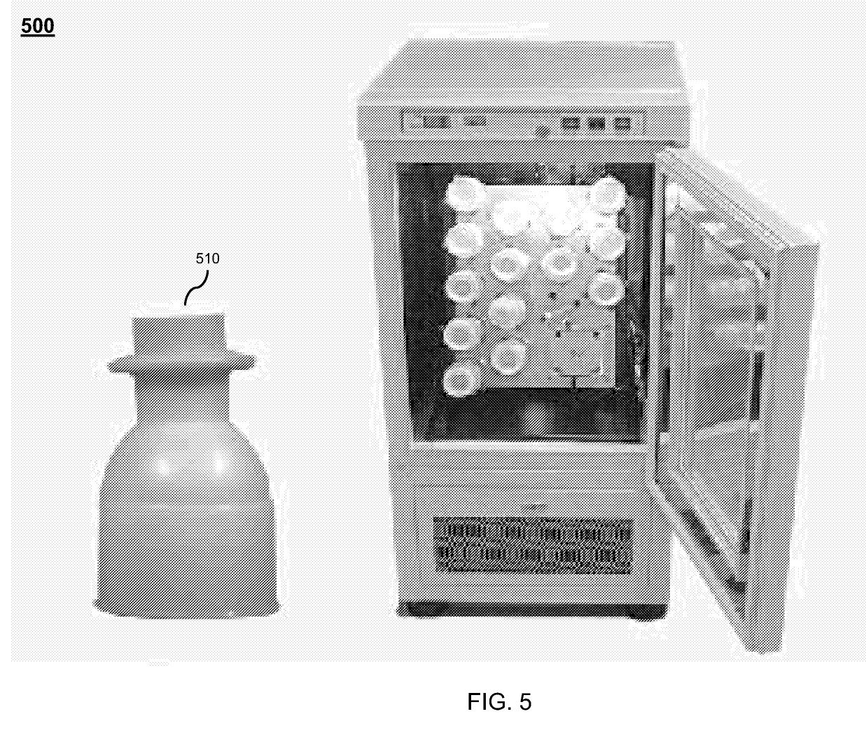

FIG. 5 shows a third embodiment of a cell culture system 500 that integrates a set of rotating bioreactors 510 into a single system. Each bioreactor in this embodiment is made of a plastic container. The bioreactors are mounted onto a housing coupled to an actuator, which can cause the bioreactors to rotate either individually, or simultaneously. The entire system can be placed in an at least partially closed environment (such as an incubator) with controlled temperature, gaseous composition, and humidity.

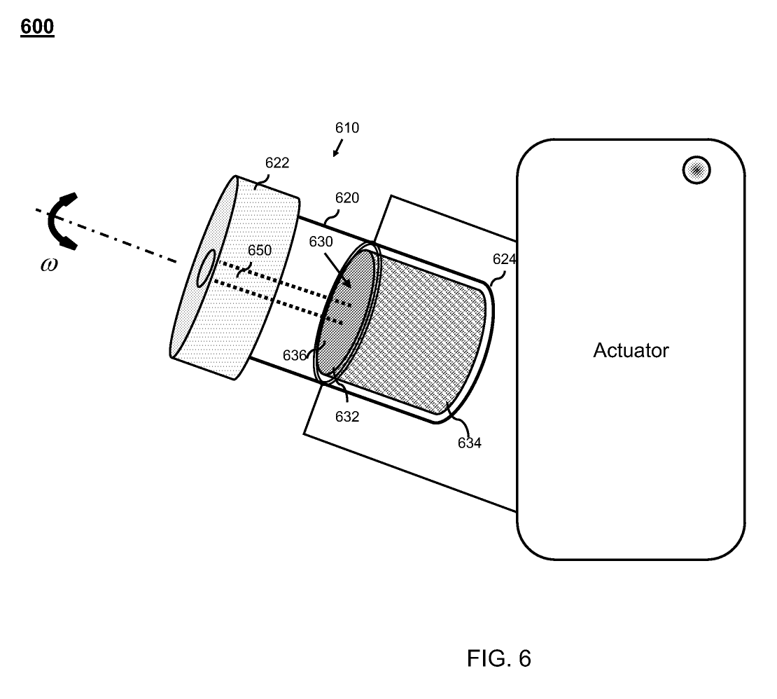

FIG. 6 shows a fourth embodiment of a cell culture system 600 that employs a rotatable bioreactor 610. This bioreactor 610 uses a two-layer structure, which includes a housing 620 having a first terminus 622 and a second close terminus 624, and a tubular member 630 disposed within the interior of the housing 620. The tubular member 630 includes an open end 632, a close end 634, and a disk member 636 coupled to the open end 632 for defining a culture chamber in which cells may grow. The disk member 636 includes at least one opening to enable exchange of a culture medium between the culture chamber and an external source.

In some examples, the tubular member 630 can be detached from the housing 620 to allow cells or tissues to be placed inside the tubular member 630. In some other examples, the tubular member 630 is fixed inside the housing 620, in which case cells or tissues can be injected into the tubular member 630 through an internal passage 650. The culture medium can be introduced through the internal passage 650 into the culture chamber. In some examples, during the rotation or rolling of the bioreactor 610, the culture medium inside the culture chamber stays relatively level.

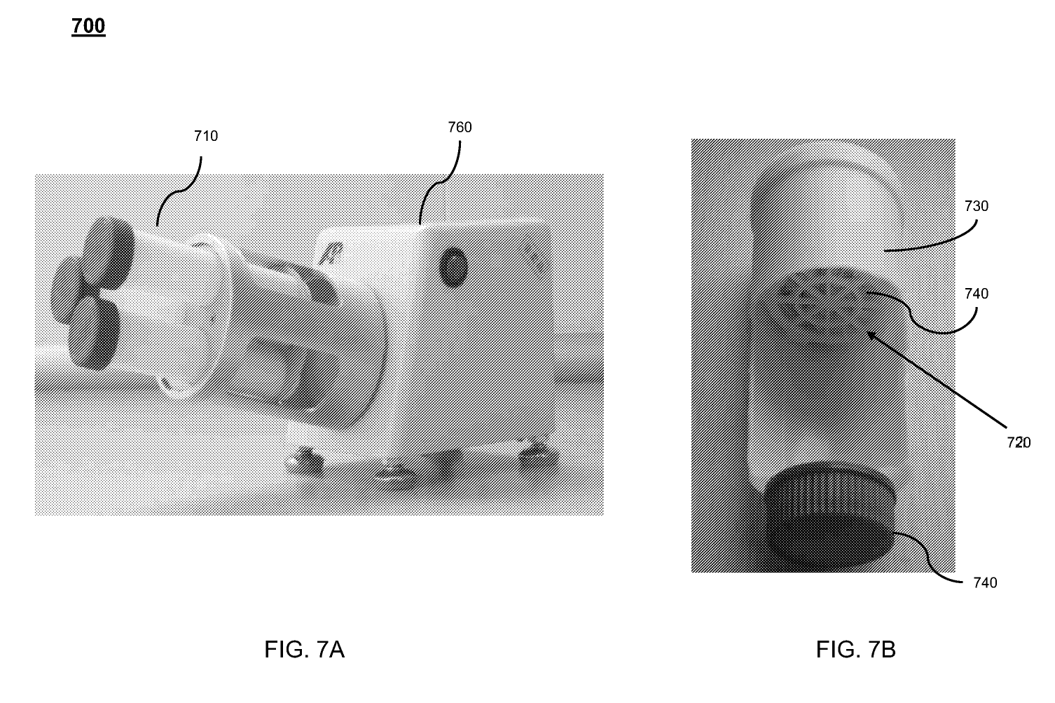

FIGS. 7A and 7B show a fifth embodiment of a cell culture system 700 that employs three rotatable bioreactors 710 coupled to a single actuator 760. Here, the bioreactor 710 includes a tubular member 730 (e.g., made of plastic materials) within which a bio-compatible matrix 740 is disposed. Examples of bio-compatible matrix include a microcarrier and a paper carrier described in an earlier section of this document. A disk member 720 is detachably disposed within the tubular member to confine the biocompatible matrix inside a culture chamber defined by the disk member 720. Depending on the particular implementation, cells or biological tissues may be introduced into the culture chamber by injection through openings in the disk member 720. In some examples, during the rotation or rolling of the bioreactor 710, the culture medium inside the culture chamber stays relatively level.

Note that some or all of the embodiments described above can be placed in a controlled environment similar to the one shown in FIG. 5.

2.2 System with “Artificial Lung”

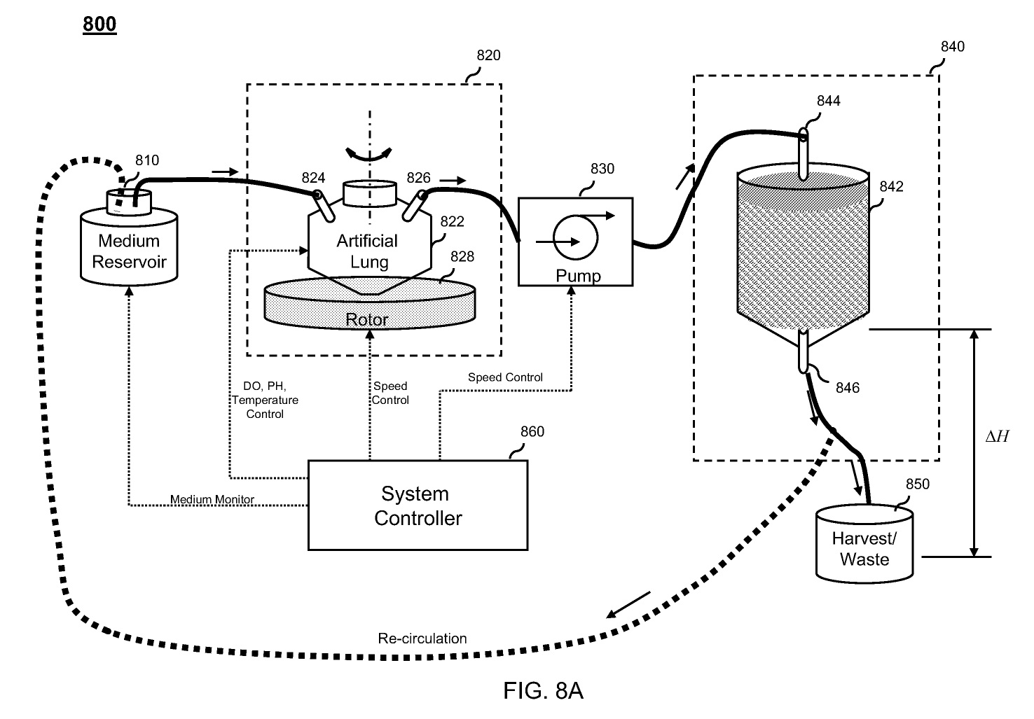

FIG. 8A shows a first embodiment of a cell culture system 800 that employs an oxygenation assembly (“artificial lung”) 820 to generate an oxygenated culture medium external to a cell culture apparatus 840.

The oxygenation assembly 820 includes a reactor 822 having an inlet 824 for receiving a culture medium provided by a medium reservoir 810 into an interior of the reactor to be oxygenated. The reactor 822 is mounted onto a rotor 828, which causes high speed orbital movement of the reactor, which in turn generates high levels of DO in the culture medium. Exemplary configurations of the reactor 822 and its operation are discussed in detail in International Patent Application Publication No. WO2007/142664A1, titled “A Method to Increase Dissolved Oxygen in a Culture Vessel,” filed September 2006, and International Patent Application Publication No. WO2006/138143A1, titled “Suspension Culture Vessels,” filed Jun. 8, 2006, the content of which is incorporated herein by reference. For example, the reactor 822 can be made of a plastic container having an inverted frusto-conical bottom or a cone-shaped round bottom. In some other examples, the reactor 822 can be implemented as a classical impellor-based deep-tank reactor.

Once being oxygenated, the culture medium inside the reactor 822 is circulated by a pump 830 (e.g., a peristaltic pump) through an outlet 826 to be provided to the cell culture apparatus 840. In one example, the cell culture apparatus 840 includes a perfusion column 842, within which a bio-compatible matrix (e.g., a paper carrier or a microcarrier) can be disposed for promoting cell growth. The original cell sources can be obtained, for example, from a bioreactor (such as the one shown in FIG. 1), or from different culture vessels, bottles, or cell factories. Cells can be seeded into the matrix using conventional techniques (e.g., via injection). To scale up cell production (e.g., for vaccine-producing cells such as Vero and MDCK), the cells attached to the matrix can be washed and trypsinized within the perfusion column, and then transferred to a different column of a larger size.

The oxygenated culture medium generated by the reactor 822 flows into an inlet 844 of the perfusion column 842, passes through the matrix to which cells are attached, and exits at an outlet 846 of the perfusion column 842 to a medium reservoir 810 or to the reactor 822 via inlet 824. As such, the medium is re-circulated through the system. In another embodiment, the medium can exist at the outlet 846 and collected to a harvest (or waste collection) 850. In some examples, the perfusion speed within the column 842 is controlled by a hydrostatic pressure caused by gravity, for example, as a function of the relative height of the column (as shown by ΔH in FIG. 8A). The higher the column 842 locates above the harvest/waste collection 850, the faster the medium exits through the column. In order to maintain a relative steady medium volume inside the column, the speed of the pump 830 is set at a suitable rate such that the medium enters into the column through inlet 844 at a speed substantially equal to the speed it exits the column though outlet 846, and the internal pressure of the column is balanced with the internal pressure of the reactor 822.

The above-described perfusion method is scalable. For example, the culture medium can be controlled to evenly perfuse into the paper carrier inside the column regardless of the size of the column. In certain applications, this feature can be desirable, especially when compared with some convention NBS perfusion bioreactors where impellor-driven culture medium may not easily get into the inside portion of the paper carrier.

In some examples, a system controller 860 is configured to monitor and control the DO level, pH level, and temperature of the culture medium inside the reactor 822 and/or the perfusion column 842. The system controller 860 can also adjust the rotation of the rotor 828 and the speed of the pump 830 to achieve a desired medium condition and a desired flow rate for cell culture.

In some examples, the culture medium exiting the perfusion column 842 does not necessarily go into the harvest/waste collection 850. Instead, it can be re-circulated back into the medium reservoir 810 to be oxygenated again by the artificial lung 820 for reuse.

FIG. 8B shows another embodiment of a cell culture system with an artificial lung. Here, the perfusion column is mounted on an adjustable stand configured for controlling the relative height of the perfusion column. The height of the perfusion column determines the flow rate of the culture medium exiting the perfusion column, based on which the controller tower can set the speed of the pump to adjust the flow-in of the oxygenated culture medium into the perfusion column at a rate substantially equal to that of the flow-out.

FIG. 8C shows a further embodiment of a cell culture system in which cells are cultured within an artificial lung, without necessarily requiring the use of an external perfusion column. The orbital movement of the artificial lung results in increased DO level in the culture medium in which cells are disposed. In some examples, a paper carrier is also placed inside the artificial lung to allow cell attachment and growth.

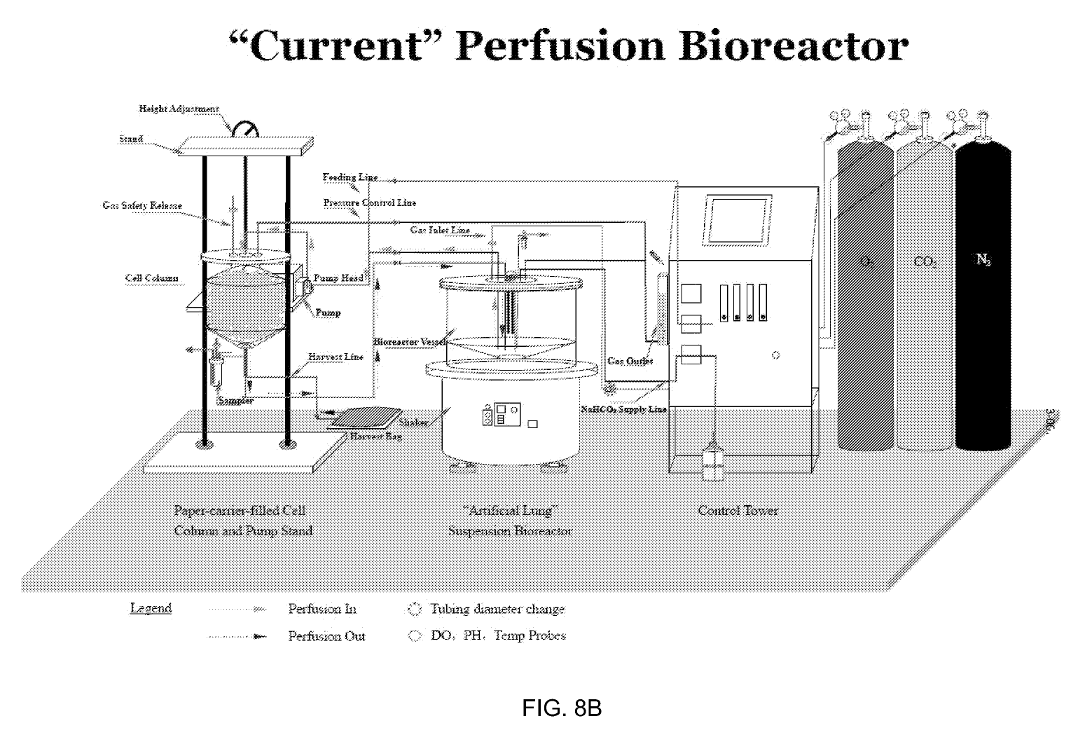

As shown in FIG. 9, the entire cell culture system 800 of FIG. 8A can be placed within a partially or completely closed environment (such as an incubator) of controlled temperature, humidity, and gaseous composition (e.g., 95% air and 5% CO₂).

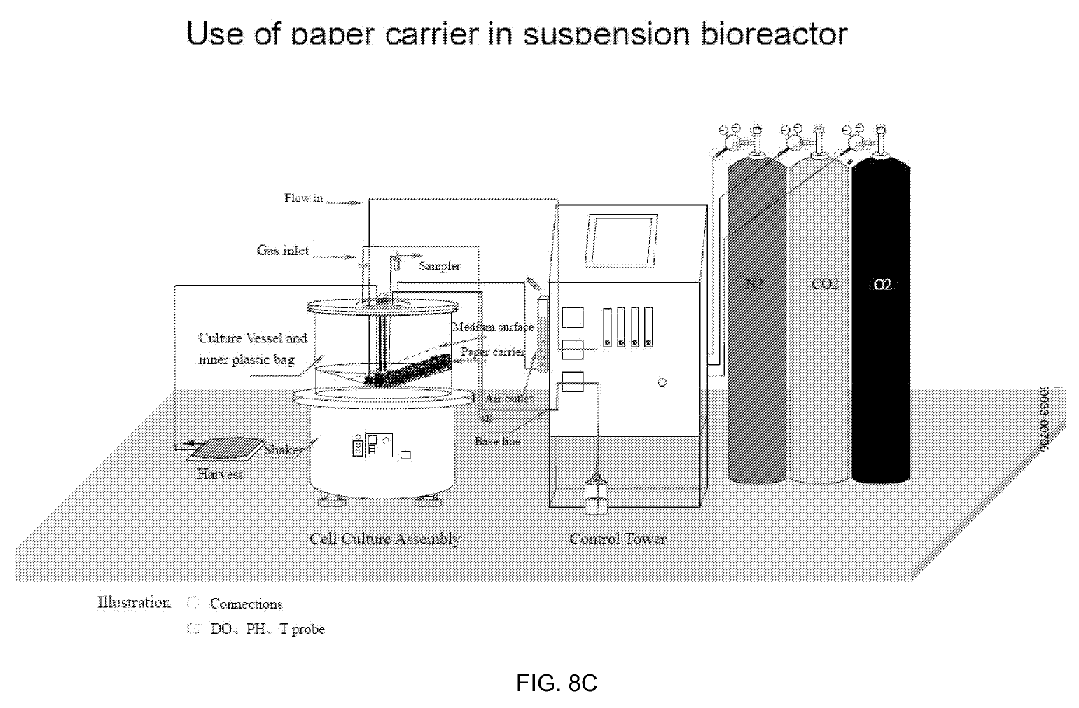

FIG. 10 shows one example of a cell culture vessel that can be used as (or inside) the perfusion column 842 of the cell culture apparatus 840. This culture vessel is made of a plastic bag filled with a paper carrier for culturing high-density adherent cells. The paper carrier is made from polymer fibers and acrylic acid glue, and shaped into a non-woven-cloth with shape, thickness, and density suitable for cell attachment and growth as well as medium circulation and perfusion. Such a system can be used, for example, to culture CHOK1 cells to produce recombinant proteins or antibodies, and to culture VERO and MDCK cells to produce vaccine.

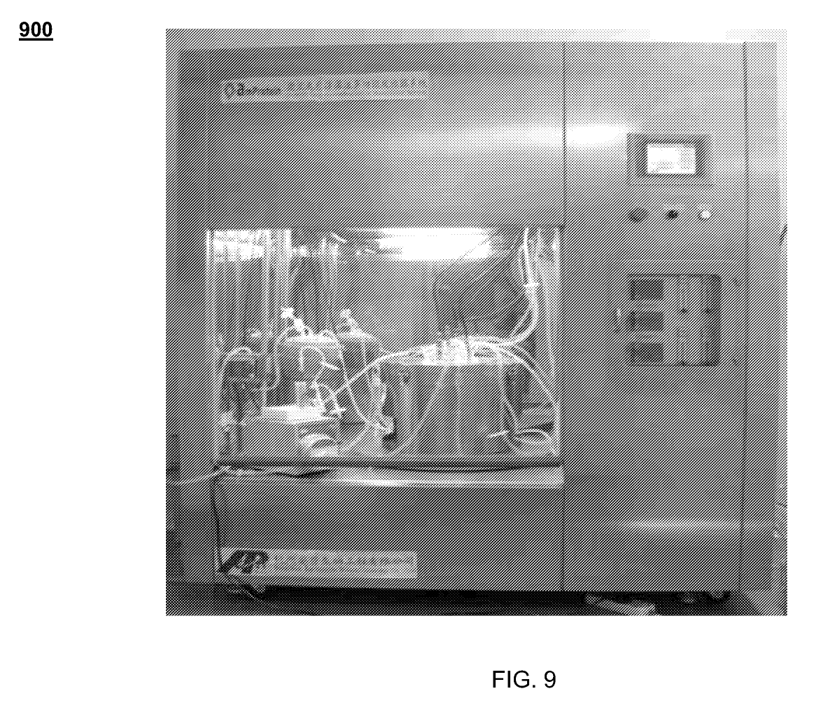

FIG. 11 shows one example of a reusable plastic bag that can be used as the reactor 822 of FIG. 8. This plastic bag has an inlet and an outlet for flow exchange, and a set of sensors (e.g., pH probe, DO probe) attached at multiple locations for monitoring the medium condition inside the bag.

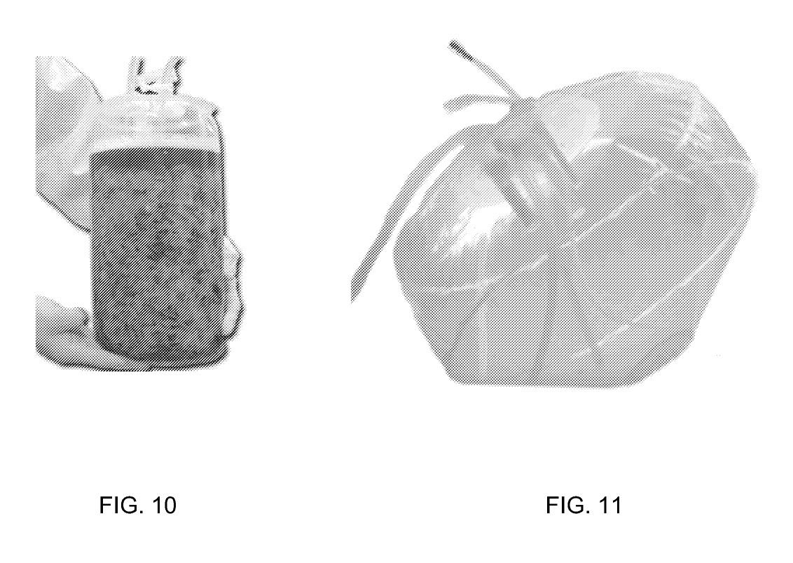

FIG. 12 shows the density distribution of cells cultured within this plastic bag of FIG. 11 that is filled with paper carrier.

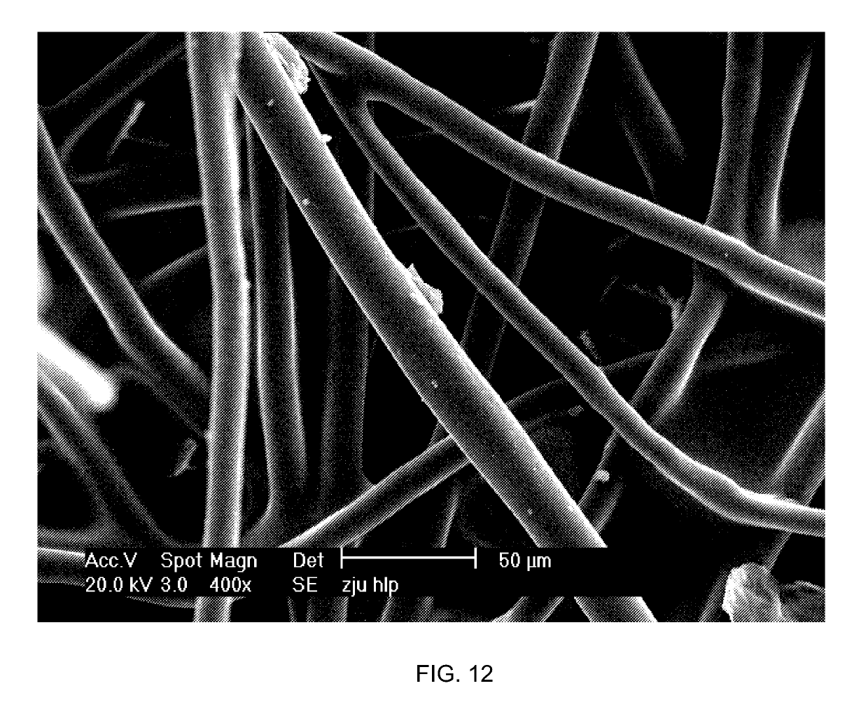

FIG. 13 shows a second embodiment of a cell culture system 900 that includes an oxygenation assembly. This oxygenation assembly is configured based on an impellor-based deep tank reactor coupled with an oxygen-sparging device. Oxygenated culture medium is circulated through a pump to into a perfusion column filled with a paper carrier.

2.3 System with a “Rotating Wheel”

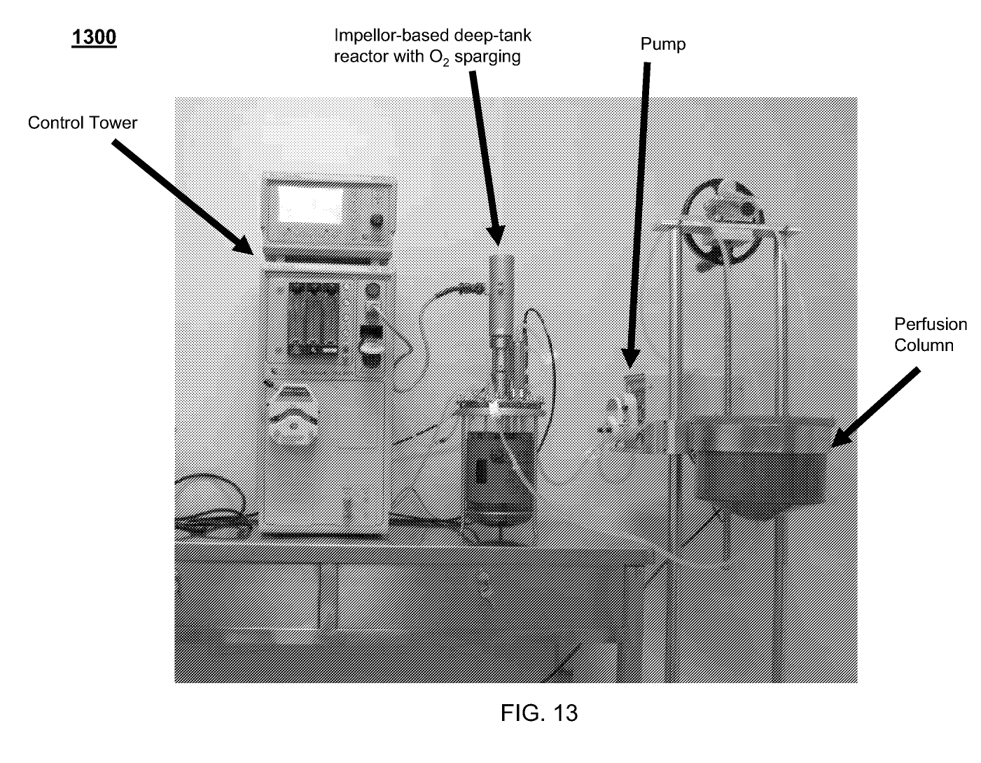

FIG. 14 shows a first embodiment of a cell culture system 1400 that uses a “rotating wheel” scheme for oxygenating a culture medium. This type of system can be applied to support both small-scale and large-scale cell culture.

The system 1400 includes a culture vessel 1410 at least partially filled with a gas (e.g., one or a combination of oxygen, carbon dioxide, nitrogen, and air). This culture vessel can be in cylindrical shape having two close ends 1412 or 1414, or configured with other types of geometry suitable for holding a cell culture medium. A rotatable shaft 1416 is positioned within the culture vessel 1410, for example, along a long axis defined by the two close ends of the culture vessel. A set of one or more disk members 1418 is concentrically mounted on the shaft 1416. The shaft 1416 can be driven by an actuator 1420 to rotate about its longitudinal axis, which in turn causes the disk members 1418 to rotate in the same manner. In this example, as the disk members 1418 are partially immersed in a culture medium, their rotation induces a relative movement of the culture medium with respect to an inner surface of the vessel 1410, which in turn accelerates the dissolution of oxygen molecules into the culture medium.

Cells can be grown in the oxygenated culture medium, for example, in suspension. The fluid conditions of the culture medium, for example, the temperature, DO level, and pH level, are monitored in real time using a set of sensors positioned inside the vessel 1410, for example, affixed to one end 1412. The measurements obtained by the sensors can be communicated, for example, in the form of electrical signals, to a control tower 1430. The control tower uses these measurement to determine the desired parameters of the system, for example, the rotational speed of the shaft 1416, the gaseous content inside the chamber, the rate of gas inflow (such as oxygen supply) if needed.

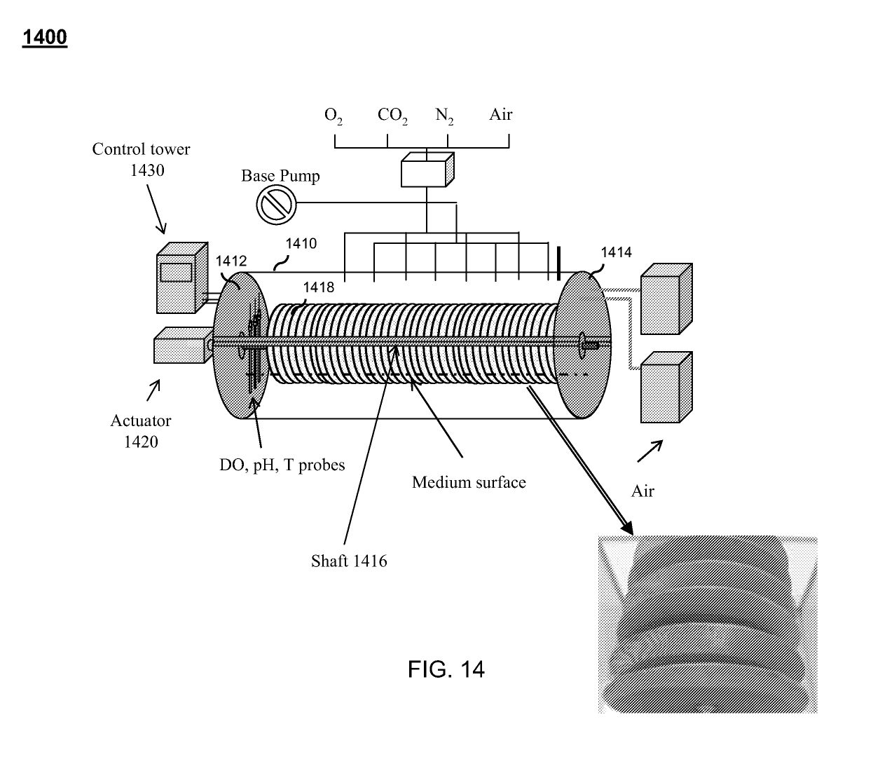

FIG. 15 shows a second and similar embodiment 1500 that also uses this rotating wheel scheme for oxygenating a culture medium. In this embodiment, cells are seeded and cultured in a bio-compatible matrix (such as a paper carrier) that is laid at the bottom of the culture vessel. This system operates in a similar manner to the one just shown in FIG. 14.

Further discussions about various embodiments of this rotating-wheel type of cell culture system are provided in U.S. Provisional Patent Application No. 61/168,740, titled “Bioreactors and Uses Thereof,” filed Apr. 13, 2009, the content of which is incorporated herein by reference.

In some examples, the wall of the culture vessel 1410 is configured to have microscopic surface features (e.g., similar to the surface features described with reference to the bioreactor 110 of FIG. 1) for improving oxygen transfer characteristics in certain applications. The wall can be made from various materials, including for example glass, silica glass, steel, aluminum, array, and different types of plastics. In one example, each disk member includes three layers of materials, including a layer of steel sheet (or alternatively organic glass sheet or plastic sheet) sandwiched between two layers of plastic or steel net.

### 3 Blood Oxygenation Systems

The following section describes various embodiments of a blood culture bioreactor for blood oxygenation as well as treatment of human diseases.

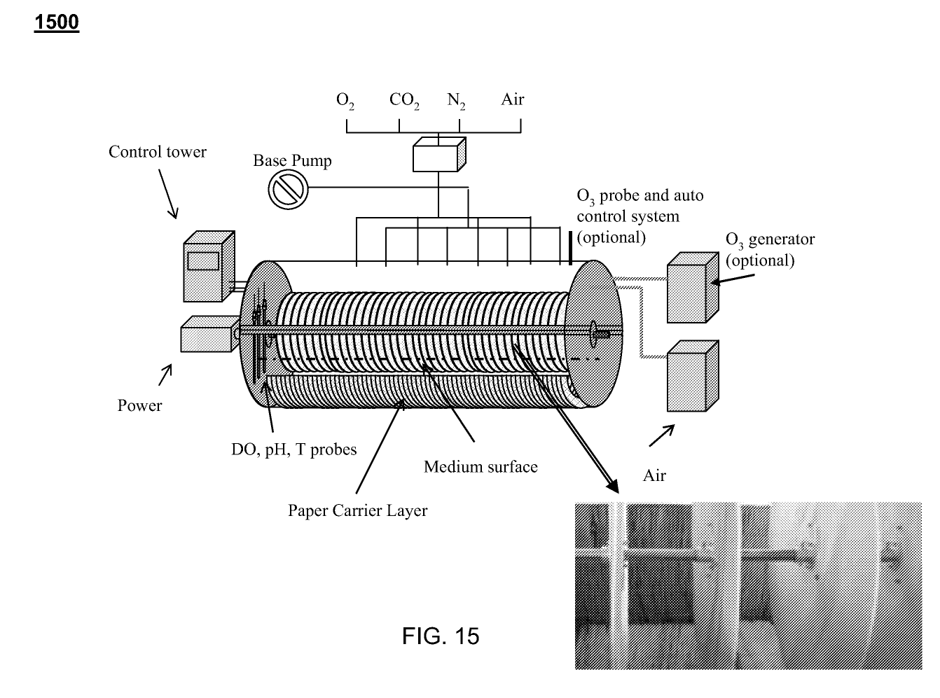

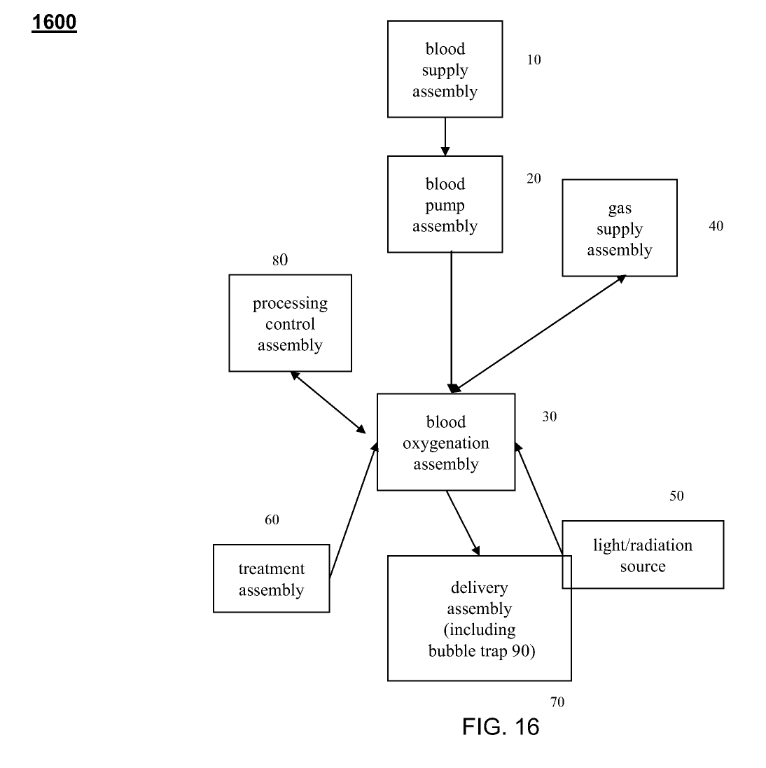

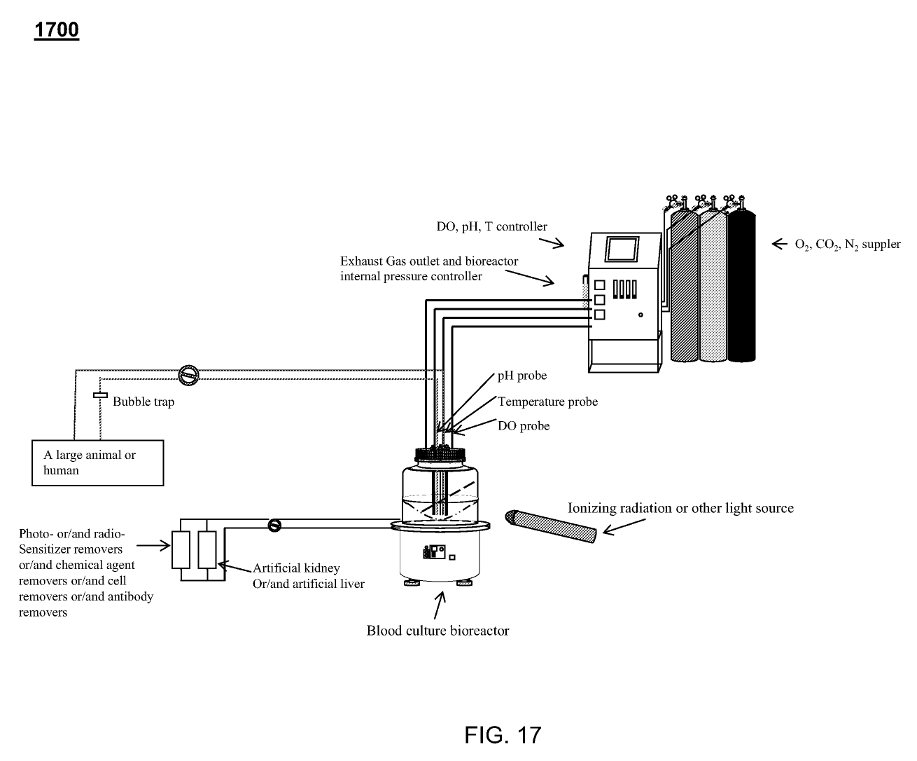

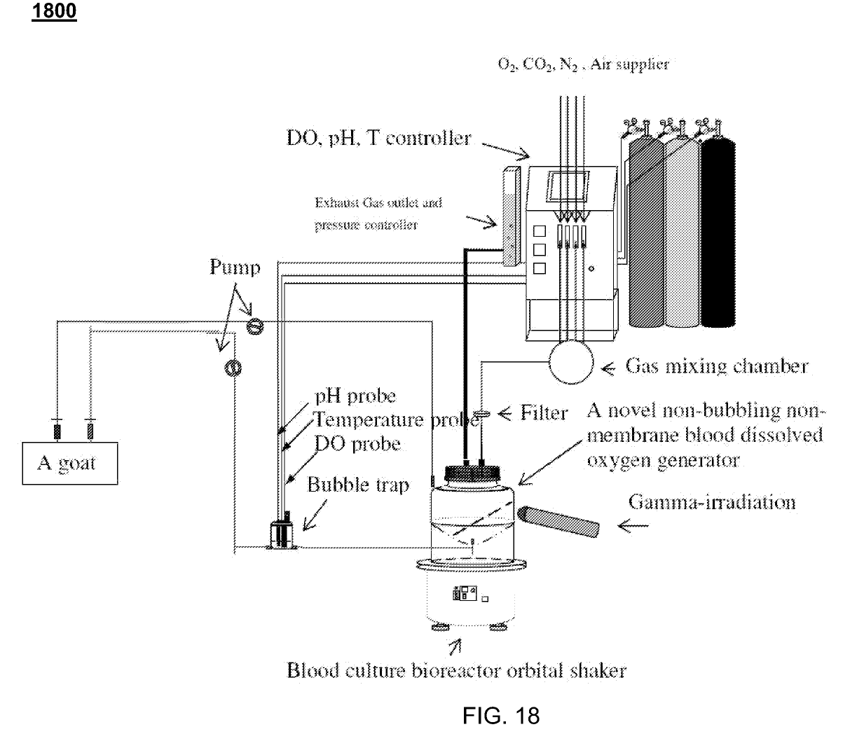

Shown in FIG. 16 is an exemplary system of extracorporeal circuit for oxygenating blood. The system includes a blood pump assembly 20, a blood oxygenation assembly 30, and a gas supply assembly 40 that is operatively coupled to the oxygenation assembly 30. Other components may include blood temperature control devices, bubble detection apparatus, pressure/temperature probes or sensors, pH probes, or pO₂ probe (shown in FIGS. 17 and 18). The various system components are operatively coupled to a processing and control assembly 80 including electronic circuitry to enable the sending and receiving of signal inputs and/or control commands amongst one or more of the various system components. A display coupled to the processing and control assembly 80 may serve as a separate user interface for the input of data and/or process control commands and/or for the display of system status and/or processing outputs. The system also includes a delivery assembly 70, which includes a bubble trap (shown in FIGS. 17 and 18). For clinical purposes, the system also includes a light/radiation source 50 and a treatment assembly 60. One application of the system in use is shown in FIG. 19.

### 3.1 Blood Oxygenation Assembly

Oxygenation of the blood takes place in the blood bioreactor/oxygenator of the blood oxygenation assembly 30. Various oxygenation methods can be used to introduce oxygen into the blood. A preferred oxygenation method is described in PCT/US06/37468 (WO2007/142664). The content of this application is incorporated herein by reference.

This method is neither sparging-based nor membrane-based conventional oxygen transfer method. It is based on dissolved oxygen (DO), namely generating microscopic bubbles in between water molecules. It takes advantage of the interaction between a material surface and a water current or other fluid (e.g., blood) current that generates dissolved oxygen or microscopic bubbles. The interaction breaks surface tension of the water or fluid current possibly at nanometer-scale level and generates microscopic air bubbles. Using this method, one can increase DO in the blood by repeatedly causing the blood to sweep or contact the air-exposed material surface with certain force.

The repeated sweeping can be generated in a housing, which defines a chamber having an inverted frusto-conical bottom or cone-shaped round bottom configured lower section as shown in FIG. 17. The oxygenation assembly can be sized depending upon the circumstances involved in a particular application.

During the sweeping, oxygen diffuses and dissolves in the fluid phase (e.g., blood) and gases from the fluid (e.g., blood) such as carbon dioxide and nitrogen may diffuse into the gas phase. The gas stream exits the blood bioreactor/oxygenator via a vent or other fluid exit conduit. In one embodiment, the vent or other fluid exit conduit is closed so as to prevent the escape of bulk gas from the oxygenation assembly.

### 3.2 Gas Supply Assembly

The assembly for supplying controlled flows or supplies of oxygen gas (e.g., gas supply assembly 40) includes a regulated source of oxygen gas, so that oxygen gas is delivered to the oxygenation assembly at a pressure greater than atmospheric pressure. Preferably, oxygen gas is supplied to the oxygenation assembly at a pressure greater than atmospheric pressure and less than about 50 p.s.i.a., the approximate maximum pressure that may be generated by commercially available blood pumps delivering blood. The assembly for supplying controlled flows or supplies of oxygen gas may be one of the many commercially available and clinically accepted oxygen delivery systems suitable for use with human patients (e.g., regulated bottled oxygen).

### 3.3 Blood Supply

The assembly for supplying controlled flows or supplies of blood (e.g., blood supply assembly 10) includes a source of blood in combination with means for providing the blood to the oxygenation assembly. The blood to be oxygenated can be blood withdrawn from a patient, so that the blood supply assembly includes a blood inlet disposed along a portion of a catheter or other similar device at least partially removably insertable within the patient's body; a pump loop that in combination with the catheter or other device defines a continuous fluid pathway between the blood inlet and the oxygenation assembly; and a blood pump for controlling the flow of blood through the pump loop, i.e., the flow of blood provided to the oxygenation assembly. The blood pump may be one of the many commercially available and clinically accepted blood pumps suitable for use with human patients.

The flow characteristics of the oxygenated blood exiting the oxygenation assembly 30 will depend upon the circumstances surrounding the particular application involved. Typically, for example, the supply of oxygenated blood provided to a catheter for infusion to a patient's body will be a controlled flow defined by the flow parameters selected by the caregiver. In an application involving the sub-selective delivery of oxygenated blood for the treatment of ischemic tissues and/or the prevention of ischemia, flow rates of about 25-100 ml/min may be advantageous. Factors influencing the determination of blood flow characteristics may include one or more of the many clinical parameters or variables of the oxygenated blood to be supplied to the catheter or to be delivered to the patient, e.g., the size of the patient, the percentage of overall circulation to be provided, the size of the blood vessel to be accessed, hemolysis, hemodilution, pO₂, pulsatility, mass flow rate, volume flow rate, temperature, hemoglobin concentration and pH.

### 3.4 Delivery Assembly

The delivery assembly includes an elongated, generally tubular assembly including a central lumen and at least one end placeable within a patient body proximate a tissue site to be treated, the end including an outlet port for the oxygenated blood. The delivery assembly includes a catheter defining a fluid pathway, including a proximal portion adapted for coupling to the oxygenated blood supply assembly, and a distal portion defining a fluid pathway removably insertable within a patient's body, for infusing the oxygenated blood to predetermined sites. Alternatively, the delivery assembly may have an infusion guidewire, sheath, or other similar interventional device of the type used to deliver fluids to patients.

### 3.5 Other Components

The system described above may include one or more gas bubble detectors, at least one of which is capable of detecting the presence of microbubbles, e.g., bubbles with diameters of about 100 μm to about 1000 μm. In addition, the system may include one or more macrobubble detectors to detect larger bubbles, such as bubbles with diameters of about 1000 μm or more. Such macrobubble detectors may include any suitable commercially available detector (e.g., those available from Transonic Inc. of New York), such as an outside, tube-mounted bubble detector including two transducers measuring attenuation of a sound pulse traveling from one side of the tube to the other. The microbubble and macrobubble detectors provide the physician or caregiver with a warning of potential clinically significant bubble generation. Such warnings also may be obtained through the use of transthoracic 2-D echo (e.g., to look for echo brightening of myocardial tissue) and the monitoring of other patient data.

The bubble detection system is able to discriminate between various size bubbles. Further, the bubble detection system operates continuously and is operatively coupled to the overall system so that an overall system shutdown occurs upon the sensing of a macrobubble. The system also may include various conventional items, such as sensors, flow meters (which also may serve a dual role as a macrobubble detector), or other clinical parameter monitoring devices; hydraulic components such as accumulators and valves for managing flow dynamics; access ports which permit withdrawal of fluids; filters or other safety devices to help ensure sterility; or other devices that generally may assist in controlling the flow of one or more of the fluids in the system. Any such devices are positioned within the system and used so as to avoid causing the formation of clinically significant bubbles within the fluid flow paths, and/or to prevent fluid flow disruptions, e.g., blockages of capillaries or other fluid pathways. Further, the system includes a biocompatible system acceptable for clinical use with human patients.

### 3.6 Application in Treating Human or Animal Disorders

As mentioned above, oxygen is a crucial nutrient for animal cells and cell damage may result from oxygen deprivation for even brief periods of time, which may lead to organ dysfunction or failure.

For example, in patients suffering from acute myocardial infarction, if the myocardium is deprived of adequate levels of oxygenated blood for a prolonged period of time, irreversible damage to the heart can result. Where the infarction is manifested in a heart attack, the coronary arteries fail to provide adequate blood flow to the heart muscle. Angioplasty or stenting of occluded vessels are often used to treat to acute myocardial infarction or myocardial ischemia. However, the procedures may cause tissue injury and are not an attractive option for some patients. To reduce the risk of tissue injury typically associated with treatments of acute myocardial infarction and myocardial ischemia, it is desirable to deliver oxygenated blood or oxygen-enriched fluids to at-risk tissues. Tissue injury is minimized or prevented by the diffusion of the dissolved oxygen from the blood or fluids to the tissue and/or blood perfusion that removes metabolites and that provides other chemical nutrients. In some cases, the treatment of acute myocardial infarction and myocardial ischemia includes perfusion of oxygenated blood or oxygen-enriched fluids.

The system described above can be used for preparation and delivery of oxygenated blood, e.g., hyperoxemic or hyperbaric blood, to a specific location within a patient's body. The system may include an extracorporeal circuit for oxygenating blood, e.g., increasing the level of oxygen in the blood, in which the blood to be oxygenated is blood withdrawn from the patient. The system also may be used for regional or localized delivery of oxygenated blood. For example, one can withdraw blood from a patient, circulate it through the above-described system to increase blood oxygen concentration, and then deliver the blood back to the patient.

The system and method describe above can be use to treat or prevent ischemia. Factors influencing the determination of blood flow characteristics for the extracorporeal circuit may include one or more of the many clinical parameters or variables of the oxygenated blood to be supplied to the patient, e.g., the size of the patient, the percentage of overall circulation to be provided, the size of the target to be accessed, hemolysis, hemodilution, pO₂, pulsatility, mass flow rate, volume flow rate, temperature, hemoglobin concentration and pH.

The system and method describe above can also be used to treat a patient having a lung failure or other respiratory disorders such as acute respiratory distress syndrome (ARDS), which interfere with oxygenation of blood. For that purpose, one can identify a subject (a human or non-human animal) having the condition and apply the procedure described herein.

The system may be used in conjunction with angiographic or guiding catheters, arterial sheaths, and/or other devices used in angioplasty and in other interventional cardiovascular procedures. The system may be used in applications involving one or more vascular openings, i.e., in either contralateral or ipsilateral procedures.

In contralateral procedures blood is withdrawn from the patient at a first location, e.g., the left femoral artery. The oxygenated blood is returned to the patient at a second location proximate the tissue to be treated. Blood oxygenation occurs as the blood pumped through the extracorporeal circuit or loop passes through an oxygenation assembly and forms the oxygenated blood to be delivered. In applications where the system includes a catheter, the catheter may include a distal end removably insertable within a patient's body through a second location, such as the patient's right femoral artery. The distal end includes at least one port in fluid communication with the central lumen and through which the oxygenated blood may exit. Further, the distal portion of the catheter may be adapted with a tip portion shaped so as to promote insertion of the device, such as through the same sheath used for interventional procedures like angioplasty, to specific predetermined locations within a patient's body. Examples of tip portion shapes which may be used include any of the standard clinically accepted tip configurations used with devices like guide catheters for providing access to and for holding in locations like the coronary ostium. Accordingly, the method may further include the step of positioning the portion of the distal end of the catheter including the fluid exit port at a predetermined location within a patient body proximate to the tissue to be treated.

In ipsilateral procedures, the system may be used along with one or more of any of a number of suitable, standard-size, clinically accepted guide catheters and/or introducer sheaths. The system, for example, may comprise a catheter, a catheter and guide catheter, or a catheter and sheath, for use within a guide catheter or introducer sheath used for the primary interventional procedure.

The present invention may also be useful in other medical applications, such as cancer therapy (e.g., the delivery of oxygen-enriched fluids directly into poorly vascularized tumors during radiation or chemotherapy treatments), neurovascular applications (e.g., the treatment of stroke and cerebral trauma patients), lung support in trauma and lung disease patients, and wound care management. Also, although the present invention may be used to raise oxygen levels, for example, in venous and arterial blood, in blood substitutes, e.g., perfluorocarbons, and in combinations thereof, for the sake of clarity and convenience reference is made herein only to arterial blood.

Further, the present invention also may be used in connection with drug fluid infusion therapies to prevent ischemia and/or to otherwise enhance the effectiveness of the therapies. Examples of drug fluids used in cardiovascular and neurological procedures which may be infused (either before, after or along with the oxygenated blood) in accordance with the present invention include, without limitation, vasodilators (e.g., nitroglycerin and nitroprusside), platelet-actives (e.g., ReoPro and Orbofiban), thrombolytics (e.g., t-PA, streptokinase, and urokinase), antiarrhythmics (e.g., lidocaine, procainamide), beta blockers (e.g., esmolol, inderal), calcium channel blockers (e.g., diltiazem, verapamil), magnesium, inotropic agents (e.g., epinephrine, dopamine), perofluorocarbons (e.g., fluosol), crystalloids (e.g., normal saline, lactated ringers), colloids (albumin, hespan), blood products (packed red blood cells, platelets, whole blood), Na+/H+ exchange inhibitors, free radical scavengers, diuretics (e.g., mannitol), antiseizure drugs (e.g., phenobarbital, valium), and neuroprotectants (e.g., lubeluzole). The drug fluids may be infused either alone or in combination depending upon the circumstances involved in a particular application, and further may be infused with agents other than those specifically listed, such as with adenosine (Adenocard, Adenoscan, Fujisawa), to reduce infarct size or to effect a desired physiologic response.

FIG. 17 shows a system in which blood is oxygenated, e.g., for delivery to a particular predetermined area within a patient's body for the treatment of conditions such as tissue ischemia and post-ischemic tissues. As shown in FIG. 17, the system includes a blood pump assembly 20 adapted for receiving a supply of blood from a blood supply assembly 10. The blood supply assembly 10 may include a supply of blood provided for infusion to a patient or to another particular desired location. The supply of blood may be received from a bag or other blood container; from a blood transferring device, such as a heart bypass system, blood oxygenator, blood filtration assembly, artificial heart and the like; from another individual; or from the patient.

The pump assembly 20 provides the blood to a blood oxygenation assembly 30. The oxygenation assembly 30 has an apparatus for raising the pO₂ of the blood, advantageously to hyperoxemic or hyperbaric levels. The oxygenation assembly 30 oxygenates blood received from the pump assembly 20. The oxygenated blood is then provided to a delivery assembly 70 for delivery to a desired location. Blood oxygenation occurs at least in part at a pressure greater than atmospheric pressure, and the oxygenated blood is delivered with a concomitant pressure drop, so that the formation of clinically significant bubbles is avoided, i.e., blood oxygenation and delivery occurs bubble-free.

What constitutes bubble-free delivery will vary depending upon the circumstances involved in a particular application. Advantageously, bubble-free delivery will occur with a complete absence of bubbles. However, in some cases of “bubble-free” delivery, one or more (perhaps maybe even thousands of) non-clinically-significant bubbles may be delivered, particularly where the gas bubbles comprise oxygen gas bubbles, which are thought to be more readily accepted by the body than bubbles of other gases. Moreover, a clinically acceptable level of bubbles in one application (e.g., a coronary procedure) might not prove to be clinically acceptable in another application (e.g., a neurological procedure).

The system shown in FIG. 17 includes a light source, which can provide ionization radiation and the system. Accordingly, the system described above can be used in rradiotherapies for curative or adjuvant treatments. For such therapies and related methods, one can use ionizing radiation in combination with radiosensitizers, ultraviolet (UVA) in combination with psoralen S-59, UVC, visible in combination with photosensitizer (such as methylene blue), filtration removal of the white blood cells, and iodine treatment followed by iodine removal, or any other means known in the art. These means can be used to kill antibiotic-resistant, virus-resistant pathogen or malignant cells. The system and related methods can be used to remove undesired immune cells, thereby reducing unwanted immune response. In some examples, r-radiation or UV treatment is used to damage/kill micro-organisms and harmful cells in the blood. After radiation, blood containing damaged or killed micro-organisms and cells can be re-circulated to the body, where those undesired substances can be removed from the blood by spleen and liver. In a preferred embodiment, post-radiation blood can be first processed using conventional laboratory techniques (such as centrifugation) to remove the undesired substances such as debris. Afterwards, such “clean” blood can re-circulated back to the body.

The system shown in FIG. 18 also includes a treatment assembly. This assembly can remove from the blood various unwanted agents, such as antibodies or chemical agents removers. Means for removing such agents are well known in the art. In one embodiment, the treatment assembly includes an apparatus for dialysis, an artificial kidney, or and artificial liver, such as those descried in U.S. Pat. Nos. 5,744,027, 7,128,836, and 7,318,892 and US Application Nos. 20080193421 and 20080045877.

The oxygenated blood preferably is provided to a patient at about 37° C. In some instances, cooling of the oxygenated blood may be desired, e.g., to induce local or regional hypothermia (e.g., temperatures below about 35° C.). For example, in neurological applications such cooling may be desired to achieve a neuroprotective effect. Hypothermia also may be regarded as an advantageous treatment or preservation technique for ischemic organs, organ donations, or reducing metabolic demand during periods of reduced perfusion.

The system described herein may include a heat exchanger assembly operable to maintain, to increase, or to decrease the temperature of the oxygenated blood as desired in view of the circumstances involved in a particular application. The temperatures for the oxygenated blood in the range of about 35° C. to about 37° C. generally will be desired, although blood temperatures outside that range (e.g., 29° C.) may be more advantageous provided that patient core temperature remains at safe levels in view of the circumstances involved in the particular application. Temperature monitoring may occur, e.g., with one or more thermocouples, thermistors or temperature sensors integrated into the electronic circuitry of a feedback controlled system, so that an operator may input a desired perfusate temperature with an expected system response time of seconds or minutes depending upon infusion flow rates and other parameters associated with the active infusion of cooled oxygenated blood. Examples of heat exchange assemblies suitable for use with the present system, either alone or integrated with a system component, include any of the numerous commercially available and clinically accepted heat exchanger systems used in blood delivery systems today, e.g., heat exchangers, heat radiating devices, convective cooling devices and closed refrigerant devices. Such devices may include, e.g., conductive/convective heat exchange tubes, made typically of stainless steel or high strength polymers, in contact with blood on one side and with a coolant on the other side.

The following specific examples are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### 4 Examples

### 4.1 Example 1

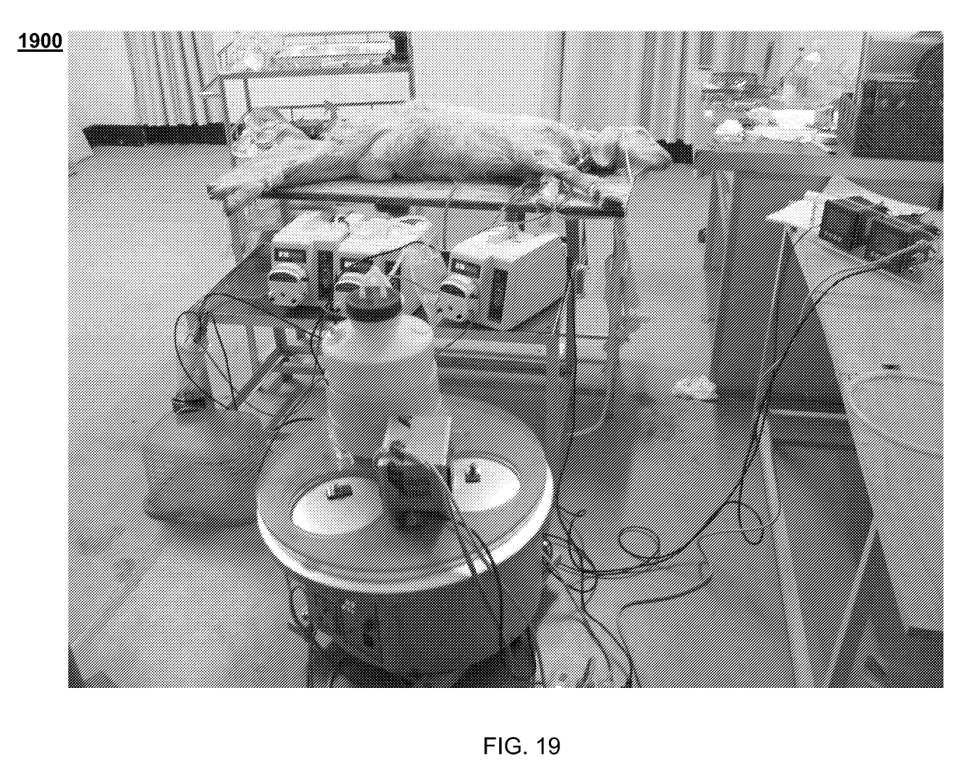

A fed-batch-culture process of CHO cells was examined using the cell culture system of FIG. 1. Specifically, cells of a CHOK1-adapted suspension cell line expressing HBsAg were cultured with microcarriers in a serum-free suspension culture medium for production phase. FIG. 20 shows the integrity and the density of microcarrier-attached CHOK1 cells cultured under an optimal DO level. The results demonstrated that the cells were cultured at a very high density without losing their integrity.

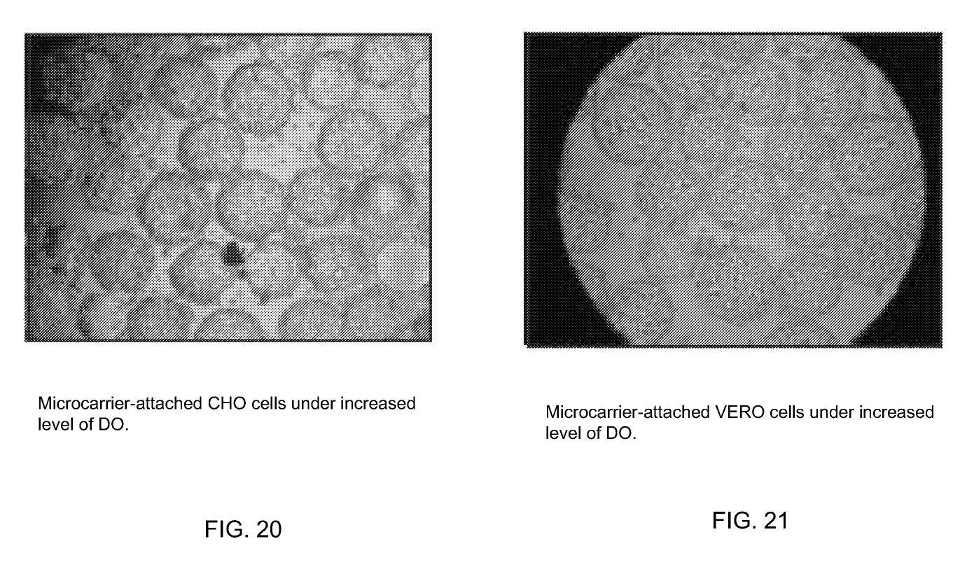

Using the same cell culture system, a fed-batch-culture process of VERO cells was examined. The cells were cultured with microcarriers in a serum-free suspension culture medium for assumed virus production phase. FIG. 21 shows the integrity and the density of microcarrier-attached VERO cells under an optimal DO level. Similar to the above-described CHO cells, the VERO cells attached to microcarriers were also cultured at a very high density while maintaining their integrity.

### 4.2 Example 2

Outside-body blood oxygen transfer and safe culture are important for life-saving medical devices. In this example, a 5-liter current suspension bioreactor was used to oxygenate goat whole blood. The current suspension bioreactor was similar to that shown in FIG. 17 or 18. Briefly, on Day 0, goat blood was collected through a vein and then cultured in the system described above in the presence of a mixture of amino acids and glucose. At Day 6, the cultured blood was returned through the vein to the same goat. The red blood cell count, white blood cell count, and platelet count were obtained on Days 0, 3, and 6 according to a standard procedure. The results are shown in Table 1 below.

TABLE 1
In vitro culturing and returning of goat whole blood (n = 3).

| 37° C. culture (day) | Day 0 | Day 3 | Day 6 |
|---|---|---|---|
| Red blood | 3.85 ± 0.2 × 10⁷ | 4.91 ± 0.3 × 10⁷ | 3.93 ± 0.2 × 10⁷ |
| cell(number/ml) | | | |
| White blood | 9.45 ± 0.4 × 10⁶ | 9 ± 0.7 × 10⁶ | 1.3 ± 0.1 × 10⁶ |
| cell(number/ml) | | | |
| Platelet(number/ml) | 9.6 ± 0.8 × 10⁶ | 7.6 ± 0.3 × 10⁶ | 7.2 ± 0.3 × 10⁶ |
| Dissolved oxygen | 100 ± 1.5% | 100 ± 2.3% | 100 ± 2.1% |
| Return of the cultured | | | vital signs normal |
| blood | | | |

Similar experiments were performed in a rabbit by using the bioreactors shown in FIGS. 6 and 7A. More specifically, blood form the rabbit were cultured in a plastic bottle having a frusto-conial bottom or in a rotating bottle before being transferred back to the rabbit. The blood cell count, white blood cell count, and platelet count were obtained and vital signs monitored. Results similar to those of the goats described above were obtained, suggesting safe culture and safe return of the cultured blood to the rabbit.

Taken together, the results indicate that the above-described current suspension bioreactors can be used as artificial lungs for oxygenating whole blood culture in vitro and returning oxygenated blood to animals.

### 4.3 Example 3

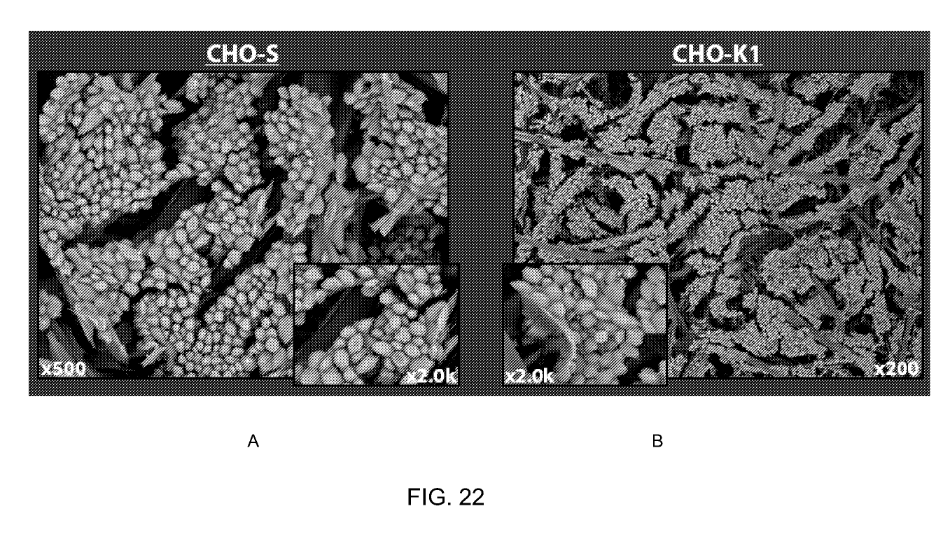

Perfusion bioreactors (FIG. 8A) were used to culture CHOK1 and CHO-S cells. The sizes of the bioreactors were 5, 50, or 150 liters. The cells were grown in high density in AmProtein's B001 medium and fed with Amprotein's F001 medium. As shown in FIG. 22, the CHOK1 and CHO-S cells were successfully cultured at very high densities while maintaining their cell integrity. Some of the results are shown in Table 2.

TABLE 2
Cells cultured at high densities in perfusion bioreactors

| | 5 L perfusion bioreactor | 50 L perfusion bioreactor | 150 L perfusion bioreactor |
|---|---|---|---|
| | (cell#/grams of paper carriers × | (cell#/grams of paper carriers × | (cell#/grams of paper carriers × |
| Cell line | total paper carrier weight 150 grams) | total paper carrier weight 1200 grams) | total paper carrier weight 3600 grams) |
| CHO-K1 | 13.7 × 10⁸ cells/g × 150 g | 16.4 × 10⁸ cells/g × 1200 g | N/A |
| CHO-S | 21.0 × 10⁸ cells/g × 150 g | 25.0 × 10⁸ cells/g × 1200 g | 18.0 × 10⁸ cells/g × 3600 g |

As shown in Table 2, the cells were cultured at very high densities and the total cell yields were also high. For example, the results indicate that the yield or productivity of a 150 L paper-carrier perfusion bioreactor equaled to that of a 1500 liter fed-batch deep tank bioreactor. The results demonstrate that the bioreactor provides a simple single-use system with stable production process.

### 4.4 Example 4

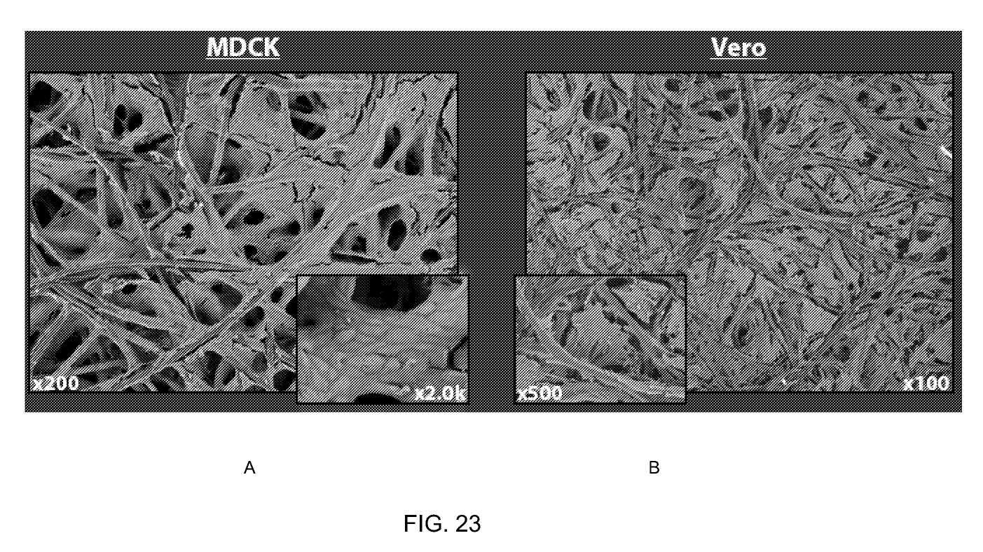

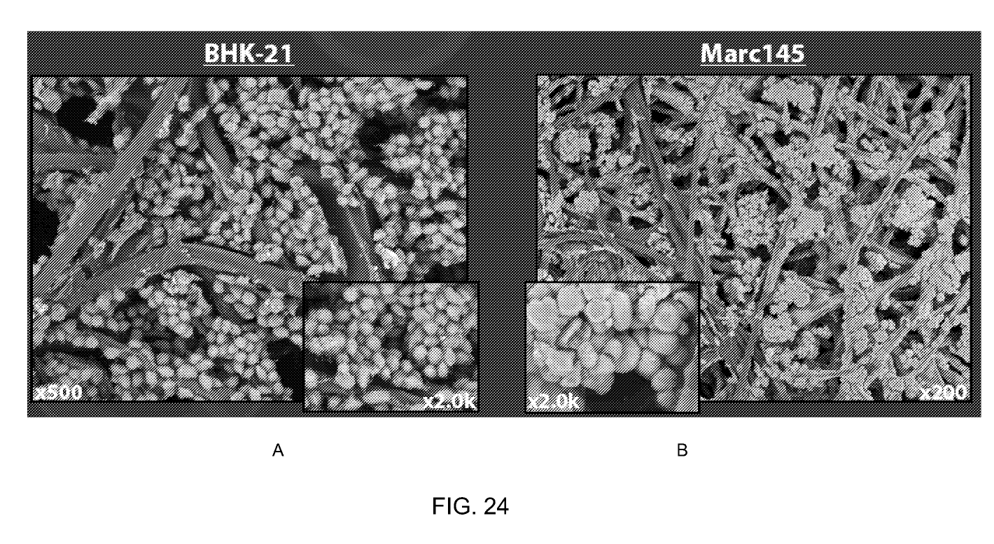

The above-described perfusion bioreactors (5 liter) were used to culture Vero, MDCK, BHK-21, Marc 145, and other cells in DMEM/F12 medium in the presence of 10% FBS in the same manner described above. As shown in FIGS. 23 and 24, the cells were successfully grown at high density in the perfusion bioreactors. Some of the results are shown in Table 3.

TABLE 3
Densities and yields of cells cultured in 5-Liter perfusion bioreactor

| | 5 L perfusion bioreactor |
|---|---|
| | (cell#/grams of paper carriers × |
| Cell line | total paper carrier weight 150 grams) |
| VERO | 6.0 × 10⁸ cells/g × 150 g |
| MDCK | 5.0 × 10⁸ cells/g × 150 g |
| Mark145 | 3.5 × 10⁸ cells/g × 150 g |
| ST1 | 4.0 × 10⁸ cells/g × 150 g |
| DF-1 (chicken) | 2.5 × 10⁸ cells/g × 150 g |
| CIK(fish) | 1.0 × 10⁸ cells/g × 150 g |
| EPC(fish) | 1.2 × 10⁸ cells/g × 150 g |

A similar experiment was performed using a 50-Liter perfusion bioreactor in which VERO cells were grown to a density of 6.5×10⁸ cells/g in a total of 1200 g of paper carrier. The results indicate that the productivity of a 50 L paper carrier perfusion bioreactor was about 1200 times that of a 20-liter conventional roller bottle used for industrial vaccine production. One advantage of such a bioreactor is the super-growth strength due to high density cell culture and inter-cell secreting growth factor support.

### 4.5 Example 5

Experiments were conducted to test the effectiveness of the above-described cell culture systems for recombinant protein production.

It was found that, for protein production, the perfusion bioreactor system worked well for high density perfusion cell culture and this system was able to culture high titer cell lines without much cell line development and process development. It was also found that, in the systems, the cells were faster to reach the production stage than conventional suspension-adapted cells.

In some experiments, a fed-perfusion culture process was examined by using a CHOK1-adapted suspension cell line expressing EPO analogue (EPO-hyperG) in a serum-free suspension culture medium for production phase. It was clearly shown that CHOK1 cells in the cell column have optimal DO level, cell density, and yield of the product (Table 4).

TABLE 4

| Cell line | Product | Culture style | Yield |
|---|---|---|---|
| CHOK1 | EPO-hyperG | Fed-perfusion | 300 mg/L |

In addition, experiments were conducted to test the culture system with the rotating bioreactor of FIG. 1 for paper-carrier based CHO cells, which expressed recombinant EGFR-6×His. In brief, 50 grams of paper-carrier was added into the rotating bioreactor (FIG. 1). The CHO cells at a density of 2×10⁶/ml were seeded and cultured for 7 days. Fresh DMEM/F12 medium was then used to replace the conditional medium. After 24 hours of culture, EGFR6×His was purified from the medium and yield was determined by standard method. It was found that the recombinant EGFR6×His yield was 53 mg/L. In contrast, in a conventional roller bottle culture, only 4 mg/L recombinant EGFR6×His purification yield was achieved.

A similar experiment was conducted using a 5-liter work volume suspension culture bioreactor (FIG. 8C). In brief, 50 grams of paper-carrier was added into a 5-liter suspension culture bioreactor vessel on a orbital shaker platform (FIG. 8C). CHO cells producing human EGFR-6×His at density of 2×10⁶/ml were seeded and cultured for 7 days in the bioreactor vessel. Fresh medium was then used to replace the conditional medium. After 24 hours of culture, recombinant EGFR6×His was purified and its yield determined in the same manner described above. It was found that the yield was 50 mg/L, which was also much higher than that of a convention roller bottle culture (4 mg/L).

### 4.6 Example 6

The blood oxygenation system shown in FIGS. 18 and 19 was used to oxygenate whole blood. Specifically, a goat was given heparin 500 units/kg body weight. The whole blood from the goat was oxygenated in vitro using the system and re-circulated back to the goat through the neck vein and artery for 8 hours. Blood pumping speed was 50 ml/minute. During the experiment, the blood pH was stabilized between 7.35-7.45 within the bioreactor of the system. Every hour, 200 units of heparin were added. It was found that blood dissolved oxygen was stabilized between 60-80%. Temperature was stably controlled at 37° C. After the re-circulating, the goat was alive and recovered to normal health. No adverse effect was observed.

### 4.7 Example 7

An experiment similar to that in example 6 was conducted to study if the above-described blood oxygenation system could be used as an artificial lung to treat partial lung functional failure.

In brief, the blood oxygenation system (FIGS. 18 and 19) was used. The pH of the blood was controlled at between 7.35-7.45 while dissolved oxygen was controlled at between 50-80%. Temperature was controlled at 37° C. The goat blood was first re-circulated at a speed of 50 ml/minute for 4 hours. Then the speed was increased to 120 ml/minute while the breathing of the goat was completely stopped by using a respiratory suppressor through vein injection. To secure its breathing blockage, the goat's respiratory track was blocked completely. Surprisingly, the goat kept its heart beating for 45 minutes. The results demonstrate that the blood oxygenation system can be effectively used as an artificial lung machine to treat patients having lung failure or similar respiratory disorders.

### 4.8 Example 8

Goat blood culture was inoculated with E. coli and cultured in the blood oxygenation system described above. Meanwhile, the inoculated culture was subject to low-dose r-irradiation treatment to remove the E. coli cells. It was found, that after the r-irradiation treatment and continuing culture, no significant E. coli proliferation was observed in the cultured blood. The while red blood cells and platelet were morphologically normal under microscope. The results suggest that the blood culture bioreactor, when combined with low-dose r-irradiation treatment, can be used to treat bacteria or virus infection in humans or non-human animals.

### 4.9 Example 9

An experiment similar to that in example 8 was also conducted. In brief, E. coli was inoculated to a goat through vein injection. After re-circulating goat blood for 9 liters (about 3× total goat blood volume) per day for 3 days together with a low-dose r-irradiation treatment, no significant E. coli proliferation in the blood culture was observed. In contrast, a control goad was inoculated with E. coli. Its blood was subject to the same procedure except the low-dose r-irradiation treatment. It was found that the control goat died at day 2. These results demonstrate the blood culture bioreactor, when combined with low-dose r-irradiation treatment, can be used to treat bacteria or virus infection in humans or non-human animals.

### 4.10 Example 10

The above-described blood re-circulating and the low-dose r-irradiation treatment (FIG. 18) were used to treat goats that had severe burn-injury. More than 20% of the total body surface of each goat was burned. The goats were treated for 7 days without antibiotic. It was found that the goat so treated survived for 7 days while a non-treated control goat died of severe infection at day 5.

### 4.11 Example 11

Experiments were conducted to remove white blood cells from the re-circulating blood of a goat.

Briefly, goat blood was obtained and cultured in the same manner described above. The goat blood within the bioreactor of the blood oxygenation system (FIG. 18) was exposed to ultraviolet light in the presence of extracorporeally administered liquid methoxsalen. Methoxsalen, a photoactive, covalently binds to DNA pyrimidine bases, cell-surface molecules, and cytoplasmic components in the exposed white cells, causing a lethal defect. These cells were then re-circulated into the goat and would die over a one-to-two-week period. During that interval, they stimulate an autologous suppressor response, in part mediated by T cells, that targets non-irradiated T cells of similar clones. The successful performance of this procedure may help human organ transplantation including bone marrow transplantation. This method is different from the classical extracorporeal photopheresis and could be used in human organ transplantation or in treating severe autoimmune diseases.

### 4.12 Example 12

The blood oxygenation system shown FIG. 18 was used in combination with γ-irradiation to remove nucleated cells from re-circulating blood of a goat.

Briefly, a goat was subject to an in vitro blood oxygenation-re-circulation procedure using the blood oxygenation system in the same manner described above. The blood from the goat was inoculated with CHO cells within the culture vessel and then exposed to low-dosage γ-irradiation so as to remove the CHO cells. After re-circulating the goat blood for 9 liters (about 3× of the total goat blood volume), no significant CHO proliferation in the blood culture was observed while non-CHO cell proliferation was observed in non-treated CHO-seeded blood culture.

The results suggest that the procedure can be used to remove nucleated cells (such as leukemia cells) from blood and therefore can be used to treat human cancer and tumor metastasis such as leukaemia, lymphoma, and sarcoma. It should be noted that this low-dosage r-irradiation does not hurt read blood cells and platelet since they do not have nuclear DNA.

### 4.13 Example 13

A rotating wheel bioreactor (FIG. 14) was used to transfer oxygen from ambient air to a CO₂-saturated PBS solution (DO=5%). The rotating speed of the wheel was 50 rpm. It was found that dissolved oxygen in the PBS increased from 5% to 100% in only 5 minutes. This result demonstrates the great oxygen transfer ability of the rotating wheel bioreactor.

The rotating wheel bioreactor was used to culture suspension cells. Specifically, a 150-liter work volume rotating wheel bioreactor (FIG. 14) was constructed and used for suspension culture (fed-batch) of CHO cells, E. coli cells, and yeast cells. Surprisingly, it was found that high cell density was routinely achieved for each of CHO cells (1.7×10⁷ cells/ml), E. coli cells (150 mg wet weight/ml), and yeast cells (42% packed cell volume). The dissolved oxygen levels at those cell densities were between 10-30% when oxygen used as a supply.

### 4.14 Example 14

The rotating wheel bioreactor (FIG. 15) was used to culture cells attached to paper-carrier. Specifically, the bioreactor was used for paper-carrier based attached culture (Fed-batch) of CHO cells, E. coli cells, and yeast cells. Unexpectedly, high cell density was routinely achieved for CHO cells (7×10⁷ cells/ml), E. coli cells (350 mg wet weight/ml), and yeast cells (62% packed cell volume). Dissolved oxygen levels at these cell densities were between 10-15% when oxygen used as a supply. In perfusion culture, high density of CHO cells of 38×10⁷/gram paper carrier was achieved. In perfusion culture dissolved oxygen level (20-80%) can be easily controlled.

### Other Embodiments

Many of the features disclosed in this specification may be combined in various combinations. Each feature disclosed in this specification may be replaced by an alternative feature serving the same, equivalent, or similar purpose.

From the above description, one skilled in the art can easily ascertain the essential characteristics of the present invention, and without departing from the spirit and scope thereof, can make various changes and modifications of the invention to adapt it to various usages and conditions. Thus, other embodiments are within the scope of the following claims.

What is claimed is:

1. A cell culture apparatus comprising:
a rotatable vessel having:
a wall for defining a culture chamber, the wall having an inner surface configured to promote gas-inner surface contact; and
an inlet formed in the wall enabling fluid communication between the culture chamber and an external fluid source; and
an actuator coupled to the vessel for rotating the vessel about a first axis to increase the level of dissolved oxygen in a fluid in the culture chamber.

2. The cell culture apparatus of claim 1, wherein the vessel has an outlet enabling fluid communication between the culture chamber and a sensor.

3. The cell culture apparatus of claim 2, further comprising the sensor for measuring a fluid condition in the culture chamber.

4. The cell culture apparatus of claim 3, wherein the sensor is configured for measuring the level of dissolved oxygen in the fluid in the culture chamber.

5. The cell culture apparatus of claim 3, wherein the sensor is configured for measuring the pH level of the fluid in the culture chamber.

6. The cell culture apparatus of claim 3, further comprising a controller for receiving a signal from the sensor and for determining the speed of rotation according to the received signal.

7. The cell culture apparatus of claim 1, wherein the external fluid source includes a liquid source and a gas source.

8. The cell culture apparatus of claim 7 further comprising a valve for selectively coupling the liquid source and the gas source to the inlet of the rotatable vessel.

9. The cell culture apparatus of claim 8, wherein the liquid source includes a culture medium.

10. The cell culture apparatus of claim 8, wherein the gas source includes one or more of the following: air, oxygen, carbon dioxide, and nitrogen.

11. The cell culture apparatus of claim 1, further comprising a housing for coupling the vessel to the actuator.

12. The cell culture apparatus of claim 11, further comprising a movable platform coupled to the housing for translating the vessel along a second axis.

13. The cell culture apparatus of claim 1, wherein the inner surface of the wall is characterized by micro-structures or nano-structures.

14. The cell culture apparatus of claim 1, wherein the vessel is disposed in an at least partially closed environment of controlled temperature or controlled gaseous composition.

15. A method for oxygenating a culture medium comprising:
introducing a culture medium into a culture chamber at least partially filled with gas, wherein the culture chamber is defined by a wall of a vessel, and an inner surface of the wall is configured to promote gas-inner surface contact; and
rotating the vessel to create a relative movement of the culture medium with respect to the inner surface of the wall of the vessel to increase the level of dissolved oxygen in the culture medium.

16. The method of claim 15, wherein the step of rotating the vessel is conducted so that microscopic bubbles carrying oxygen molecules are generated.

17. The method of claim 15, further comprising:
measuring a fluid condition of the culture medium; and
controlling a speed of rotation according to the measured fluid condition.

18. The method of claim 17, wherein the fluid condition includes one or more of the following: a pH level, a dissolved oxygen level, a carbon dioxide level, and a temperature.

19. A culture system comprising:
an oxygenation apparatus configured for oxygenating a culture medium, the oxygenation apparatus including:
a vessel having an inlet for receiving the culture medium to be oxygenated, an outlet for providing oxygenated culture medium, and a wall with an inner surface configured to promote gas-inner surface contact; and
an actuator coupled to the vessel for driving the vessel in orbital movement to increase the level of dissolved oxygen in the culture medium; and
a culture apparatus configured for receiving the oxygenated culture medium from the oxygenation apparatus, the culture apparatus including:
a port for receiving a biological material into an interior of the culture apparatus; and
an inlet in fluid communication with the interior of the culture apparatus to deliver the oxygenated culture medium into contact with the biological material.

20. The culture system of claim 19, wherein the biological material includes a group of cells.

21. The culture system of claim 19, wherein the culture apparatus includes a perfusion column.

22. The culture system of claim 21, wherein the flow rate of the culture medium exiting the perfusion column is controlled based on a height of the perfusion column.

23. The culture system of claim 22, wherein the flow rate of the culture medium entering the perfusion column is controlled to be substantially equal to the flow rate of the culture medium exiting the perfusion column.

24. The culture system of claim 23, wherein the perfusion column is mounted on an adjustable stand configured for adjusting the height of the perfusion column.

25. The culture system of claim 21, wherein the perfusion column is configured to receive a bio-compatible matrix suitable for cell culture.

26. The culture system of claim 25, wherein the bio-compatible matrix includes a paper carrier having woven or non-woven polymer fibers.

27. The culture system of claim 19, further comprising a pump for generating a flow of the oxygenated culture medium to be received by the culture apparatus.

28. The culture system of claim 27, further comprising a controller for controlling a speed of the flow of the oxygenated culture medium.

29. The culture system of claim 28, wherein the controller is further configured to control a rotational or an orbital movement speed of the vessel of the oxygenation apparatus.

30. The culture system of claim 19, wherein the vessel of the oxygenation apparatus has an inverted frusto-conical bottom or cone-shaped round bottom.

31. A culture system comprising:
a housing having a first terminus and a second and close terminus opposed to the first terminus, the first terminus having a port for receiving and dispensing a culture medium;
a tubular member disposed within an interior of the housing, the tubular member having:
a first open end;
a second and close end positioned at the second terminus of the housing; and
a disk membrane coupled to the first open end for at least partially enclosing an interior of the second tubular member to define a culture chamber, wherein the disk member includes at least an opening enabling fluid communication between the culture chamber and an exterior of the second tubular member; and
an actuator coupled to the housing for rotating the housing to increase the level of dissolved oxygen in a fluid in the culture chamber.

32. The culture system of claim 31, wherein the tubular member is detachably disposed within the interior of the housing.

33. The culture system of claim 31, wherein an inner surface of the tubular member is configured to promote gas-inner surface contact.

34. The culture system of claim 31, wherein the tubular member is configured to receive a bio-compatible matrix suitable for cell culture through its first open end.

35. A cell culture apparatus comprising:
a tubular member having a first end and a second and close end opposed to the first end, the first end having a port for receiving and dispensing a culture medium;
a bio-compatible matrix disposed within an interior of the tubular member; and
a disk member detachably disposed within the interior of the tubular member for substantially confining the bio-compatible matrix within a culture chamber defined between the disk member and the second end of the tubular member;
wherein the disk member includes at least an opening for permitting a flow of the culture medium into the culture chamber.

36. The cell culture apparatus of claim 35, further comprising a cap coupled to the first end of the tubular member for sealingly enclosing the interior of the tubular member.

37. A cell culture system comprising:
an elongated member having:
a first close end;
a second close end opposed to the first close end;
a wall defining a culture chamber at least partially filled with gas; and
an inlet formed in the wall capable of receiving a culture medium;
a shaft positioned substantially within the culture chamber, the shaft having a first end and a second end respectively positioned at a corresponding end of the elongated member;
a set of one or more disk elements concentrically mounted on the shaft;
an actuator mechanically coupled to the shaft for rotating the disk elements about a longitudinal axis of the shaft for inducing a relative movement of the culture medium with respect to an inner surface of the wall of the elongated member; and
a sensor for detecting the level of dissolved oxygen in the culture medium contained in the culture chamber.

38. The cell culture system of claim 37, further comprising a bio-compatible matrix disposed at a lower section of the interior of the elongate member.

39. The cell culture system of claim 38, wherein the bio-compatible matrix includes polymer fibers.

40. The cell culture system of claim 37, further comprising a controller for receiving a signal from the sensor indicative of the level of dissolved oxygen in the culture medium.

41. The cell culture system of claim 40, wherein the controller is further configured for controlling a speed of rotation of the shaft based on the received signal from the sensor.

42. The cell culture system of claim 41, wherein the elongated member further includes a second inlet for introducing a flow of gas into the interior of the elongated member.

43. The cell culture system of claim 42, wherein the controller is further configured for controlling a gaseous composition in the interior of the elongated member.

44. A system for oxygenating blood and delivering the oxygenated blood to a subject, the system comprising:
(1) a blood pump assembly adapted for coupling to a supply of blood;
(2) a blood oxygenation assembly coupled to the blood pump assembly, the blood oxygenation assembly receiving the blood from the blood pump assembly and oxygenating the blood, the blood oxygenation assembly comprising a blood bioreactor/oxygenator, wherein the blood bioreactor/oxygenator comprises:
(a) a housing including a wall;
(b) a first inlet in the housing adapted to deliver oxygen-containing gas stream into contact with a first surface of the wall;
(c) a second inlet in the housing adapted to deliver the blood into contact with a second surface of the wall, wherein the blood bioreactor/oxygenator allows a movement of the blood so that the blood contacts with the first surface to form oxygenated blood; and
(d) a first outlet and a second outlet in the housing for expelling CO2-containing gas and the oxygenated blood, respectively;
(3) a delivery assembly coupled to the second outlet, the delivery assembly being adapted to deliver the oxygenated blood to the subject; and
(4) a control assembly coupled to the blood oxygenation assembly.

45. The system of claim 44, wherein the blood bioreactor/oxygenator allows the movement of the blood so that the blood and the oxygen-containing gas repeatedly and consecutively contact the first surface.

46. The system of claim 45, wherein the movement is a circular or orbital movement.

47. The system of claim 44, wherein the housing defines a chamber that includes an inverted frusto-conical bottom or cone-shaped round bottom configured lower section.

48. The system of claim 44, the blood bioreactor/oxygenator further comprises a pH probe, a temperature probe, or a DO probe.

49. The system of claim 44, wherein the system further comprises a treatment assembly, wherein the treatment assembly kills or removes an unwanted agent from the blood.

50. The system of claim 49, wherein the unwanted agent is a chemical agent, a bio molecule, an antibody, a microbial cell, a virus, or a cell.

51. A method for oxygenating blood of a subject comprising using the system of claim 44.

52. A method for removing an unwanted agent from the blood of a subject comprising using the system of claim 44.

53. The method of claim 52, wherein the subject has a cancer and the unwanted agent is a tumor cell.

54. The method of claim 52, wherein the subject has an infection and the unwanted agent is a bacterial cell, a yeast, or a virus.
